SEQUENCE LISTING (4 sequences)

SEQ ID NO: 1 - DNA, 301 bp, Sus scrofa
gccaccagcatgagactccaccgcctcctccttgtattcctcctcatggtcctgttacctgtgccaggtctactaaaaaacataggaaattctgttagctgcttaaggaataaaggcgtgtgtatgccgggcaagtgtgctccaaagatgaaacagatcggcacctgtggcatgccccaagtcaaatgctgcaaaaggaagtaaaaagaaagtgaagaaacaaccccacagatatggctcagaagctgctcccttggaaaagcatataaaatttaaactagattaaatctttgttcaaggc

SEQ ID NO: 2 - protein, 64 aa, Sus scrofa
MRLHRLLLVFLLMVLLPVPGLLKNIGNSVSCLRNKGVCMPGKCAPKMKQIGTCGMPQVKCCKRK

SEQ ID NO: 3 - DNA, 24 bp, Artificial
  PCR Sense Primer
tcccatgagactccaccgcctcct

SEQ ID NO: 4 - DNA, 27 bp, Artificial
  PCR Antisense Primer
ttcgagcagcttctgagccatatctgt

(19) United States
(12) Patent Application Publication
(10) Pub. No.: US 2006/0008466 A1
(43) Pub. Date: Jan. 12, 2006

(54) Methods for treating and preventing microbial infections

(75) Inventors: Shokrollah Elahi, Saskatoon (CA); Volker Gerdts, Saskatoon (CA); Lorne Babiuk, Saskatoon (CA)

Correspondence Address: ROBINS & PASTERNAK, 1731 EMBARCADERO ROAD, SUITE 230, PALO ALTO, CA, 94303, US

(73) Assignee: University of Saskatchewan, Saskatoon (CA)

(21) Appl. No.: 11/127,061

(22) Filed: May 10, 2005

Related U.S. Application Data
(60) Provisional application No. 60/586,169, filed on Jul. 7, 2004.

(51) Int. Cl. A61K 39/02; A61K 38/16

(52) U.S. Cl. 424/190.1; 514/12

(57) ABSTRACT

Methods for the treatment and prevention of microbial infection, such as infections caused by bacteria, viruses, fungi and parasites are disclosed, as are adjuvants for use with vaccines against such microbes. The methods use porcine β-defensins, such as porcine β-defensin-1 and are particularly useful for treating or preventing infections caused by gram-negative bacteria, such as pertussis.

### CROSS-REFERENCE TO RELATED APPLICATION

This application claims benefit under 35 U.S.C. § 119(e) of provisional application 60/586,169 filed on Jul. 7, 2004, which application is hereby incorporated by reference in its entirety.

### TECHNICAL FIELD

The present invention pertains generally to methods for treating and preventing microbial infections. In particular, the invention relates to the use of porcine β-defensins for the treatment and prevention of infections caused by bacteria, viruses, fungi and parasites.

### BACKGROUND

Antimicrobial peptides (AMPs), also called “host defense peptides” or “cationic peptides” represent crucial elements of the innate immune system. AMPs can be classified into two broad groups of either cyclic or linear peptides which include a wide variety of molecules such as lysozymes, lactoferrin, secretory leukoprotease inhibitor, defensins and cathelicidins. Typically, AMPs are small molecules which often display a strong cationic charge. AMPs act as effector molecules of innate immunity by killing a broad spectrum of microbes including bacteria such as Gram-positive bacteria, Gram-negative bacteria, fungi, parasites and viruses.

Defensins are the most widely studied family of AMPs. Defensins are particularly interesting as they display a plethora of immunomodulatory activities, including the ability to stimulate chemotaxis of immature dendritic cells and T-cells, glucocorticoid production, macrophage phagocytosis, mast cell degranulation, complement activation and IL-8 production by epithelial cells (Yang et al., Cell. Mol. Life Sci. (2001) 58:978-989). Thus, defensins represent an important link between innate and acquired immunity and are potent immune modulators and adjuvants for vaccines. For example, low concentrations of a human α-defensins (10-100 ng, administered with KLH absorbed to alum) lead to strong augmentation of IgG1, IgG2a and IgG2b, indicative of stimulation of both Th1 and Th2 responses (Tani et al., Int. Immunol. (2000) 12:691-700; Lillard et al., Proc. Natl. Acad. Sci. USA (1999) 96:651-656). In contrast, α- and β-defensins, co-delivered intranasally, have been reported to stimulate primarily a Th-2 response (IgG1 and IgG2b, but not IgG2a or IgM) to ovalbumin (Brogden et al., Int. J. Antimicrob. Agents (2003) 22:465-478). Intradermal immunization of mice with a fusion construct encoding the HIV glycoprotein 120 and β-defensin 2 resulted in strong humoral and cell-mediated mucosal immune responses against HIV and antitumor immune responses were greatly enhanced by the presence of defensins.

AMPs are also involved in controlling bacterial invasion at the mucosal surfaces. AMPs are expressed by epithelial cells of the mucosal and the cutaneous surfaces, by neutrophils, and in some species, by macrophages. AMPs form part of the “permeability barrier” of the skin and the gut and it is thought that deficiency in expression of AMPs is linked to Crohn's disease (Fellermann et al., Eur. J. Gastroenterol. Hepatol. (2003) 15:627-634; Schmid et al., Z. Gastroenterol. (2004) 42:333-338; Wehkamp et al., Inflamm. Bowel Dis. (2003) 9:215-223), and ulcerative colitis or infections with Helicobacter pylori (Bajaj-Elliott, M., Gut (2003) 52:166-167; Bajaj-Elliott et al., Gut (2002) 51:356-361; Uehara et al., J. Med. Microbiol. (2003) 52:41-45). In addition, the potential of xenodefensins (derived from other species) for disease protection has been demonstrated. In particular, when the human α-defensin 5 (hBD-5) is transgenically expressed in intestinal Paneth cells of mice, complete protection against a lethal challenge with Salmonella typhimurium results, highlighting the importance of defensins for disease protection at the mucosal surface (Ganz, T., Science (2002) 298:977-979; Salzman et al., Nature (2003) 422:522-526). Thus, defensins represent powerful immune modulators which have both a direct and an indirect antimicrobial effect by enhancing the body's immune response to the invading pathogens.

Porcine β-defensin-1 (“pBD-1”) (Zhang et al., J. Biol. Chem. (1999) 274:24031-24037; Zhang et al, FEBS Lett. (1998) 424:37-40) has been isolated from porcine tissues and is a member of the β-defensin family. The cDNA sequence of this cationic peptide encodes a 64 amino acid prepro-peptide, which contains the β-defensin consensus sequence of six invariantly spaced cysteine residues. The expression of pBD-1 is regulated developmentally and increases postnatally throughout the epithelia of the respiratory and gastrointestinal tracts. Furthermore, pBD-1 can be detected in nearly all organs and cells of pig including thymus, spleen, urinary bladder, lymph node, brain, liver, kidney, testis, skin, heart, muscle, bone marrow, alveolar macrophages, peripheral blood neutrophils and the umbilical cord. The only cells that do not express pBD-1 are peripheral blood mononuclear cells (Zhang et al., J. Biol. Chem. (1999) 274:24031-24037). Although, pBD-1 is expressed throughout the epithelia of different organs, the highest level of pBD-1 mRNA is detected in tongue epithelial tissues. The location of this defensin-rich layer in the buccal mucosa could potentially function as a cellular antimicrobial barrier that prevents clinical infections despite its frequent exposure to trauma and microtrauma during mastication. The salt-dependent bactericidal activity of pBD-1 (40 μg/ml) has been demonstrated against both Gram-positive and Gram-negative bacteria such as Escherchia coli, Salmonella typhimurium, Listeria monocytogenes, and Candida albicans (Shi et al., Infect. Immun. (1999) 67:3121-3127).

pBD-1 displays significant homology to twelve bovine β-defensins (LAP; EBD; and BNBD-1, 3, 4, 5, 6, 7, 9, 10, 12), two ovine beta-defensins (sBD-1 and sBD-2), and the human beta defensin-2. However, pBD-1 most closely resembles bovine and sheep β-defensins in its expression pattern, cDNA and prepro-peptide sequences. The wide expression of pBD-1 throughout the body even in those organs that do not directly interfere with a microbial environment, suggests that it may contribute to both mucosal and systemic host defenses in pigs and may have other functions beyond its antimicrobial activity.

Pertussis (whooping cough) is an acute infection of the respiratory tract caused by Bordetella pertussis and occasionally by B. parapertussis (reviewed in von Konig et al., Lancet Infect. Dis. (2002) 2:744-750). Bordetella are Gram-negative pleiomorphic aerobic bacteria. Infection by B. pertussis is initiated by attachment to the ciliated epithelial cells of the nasopharynx and mediated by surface adhesions including pertactin, filamentous hemagglutinin, and fimbriae. Following attachment, the organism multiplies and produces a variety of toxins that cause local mucosal damage (pertussis toxin, tracheal cytotoxin, dermatonecrotic toxin). The disease is characterized by a prolonged cough. Clinical symptoms including seizures, encephalopathy, and pneumonia and vary by age. Importantly, clinical manifestations of pertussis are more severe in infants and young children. In the developed world, nearly all mortality occurs in infants too young to have been immunized or in unimmunized individuals. However, not all children develop classical disease. In infants, the illness may be atypical; often apnea and cyanosis are the only symptoms at presentation. Additionally, seizures, encephalopathy, and pneumonia are all more common in young infants.

Currently, the preferred method of controlling pertussis in infants and young children is by immunization using either a whole cell (wP) or acellular vaccine (aP) (see, e.g., Wirsing et al., Lancet (2002) 2:744-750; Edwards et al., JAMA (1993) 269:53-56; Lin et al., Vaccine (1997) 15:917-921; Rothstein et al., Vaccine (1999) 17:2996-3006; Keitel et al., J. Infect. Dis. (1999) 180:397-403; Halperin et al., Vaccine (2000) 18:1312-1319; Tunbull et al., Vaccine (2001) 19:628-636). Vaccination with either the wP or aP has significantly reduced the incidence of disease. Immunization against pertussis routinely consists of three doses (aP) given at 2, 4 and 6 months of age, a fourth dose at 18 months of age and a fifth dose at 4 to 6 years of age. When more rapid protection is preferred, the first three doses may be administered at intervals of 4 weeks and the fourth dose given as soon as 6 months after the third dose. All combined aP vaccines are adsorbed vaccines and must be given intramuscularly.

Because adverse reactions are more common and the disease is typically less severe in older children, adolescents and adults, immunization with the whole-cell pertussis vaccine is not recommended for people seven years of age or older. However, pertussis in this group is an important source of infection for young infants. For this reason, studies are underway to assess the role of pertussis in adolescents and adults with cough illness, and the safety, immunogenicity and efficacy of acellular pertussis vaccine in these age groups. Ensuring the complete immunization of all children remains the most important preventive measure in maximizing control of pertussis.

Acquired immunity against pertussis develops after natural infection and confers relatively long-lived protection against subsequent infection. Infection usually results in potent induction of CD4⁺ T helper (Th) type 1 T cells, and it is believed that these cells are required for disease protection. A large body of data also indicates that antibodies against pertussis toxin (PT), filamentous hemagglutinin (FHA), pertactin (PRN) or adenylate cyclase toxin (ACT) play a role in protection. However, these titers often do not correlate with protection in large clinical trials and fail to protect against subclinical infection. Additionally, these data are mostly based on serological ELISA assays which do not reflect the functional relevance of these antibodies. Evidence clearly indicates that circulating serum antibodies are important for protection, but it is not clear if this effect is a result of transsudation to the mucosal surfaces of the lung. Recent studies, however, have demonstrated the importance of mucosal immunity in disease prevention, but so far no vaccines have been developed for stimulating the mucosal immune response.

Innate defense mechanisms against pertussis include interferon-γ (IFN-γ) which is required for confining the bacteria to the respiratory tract at an early stage of infection. IFN-γ upregulates release of antimicrobial components including AMPs in macrophages and neutrophils. In vitro studies have revealed that B. pertussis isolates display a broad spectra of susceptibilities to various AMPs including cecropin, magainine-II-amide, protamine, and melittin (Fernandez et al., Antimicrob. Agents Chemother. (1996) 40:1041-1043; Baneman et al., Infect. Immun. (1998) 66:5607-5612). However, no effect was demonstrated for human β-defensin-1 (Baneman et al., Infect. Immun. (1998) 66:5607-5612).

Disease models to test vaccines against pertussis are currently restricted to the aerosol and intracerebral challenge model in mice. As a result, very little is known about the immune response against B. pertussis at the mucosal surfaces and the role of maternal antibodies in disease protection. Moreover, to date, there have been no studies regarding the use of pBD-1 in the prevention or treatment of pertussis infection.

The wide-spread availability of a safe and effective method to prevent infectious diseases, such as pertussis, would be highly desirable and could save a considerable number of lives.

### SUMMARY OF THE INVENTION

The present invention provides a simple, accurate and efficient method for treating and preventing infection caused by a variety of microorganisms, including Gram-negative and Gram-positive bacteria, viruses, fungi and parasites. The invention also provides a method for enhancing the immune response to a vaccine administered against these microorganisms.

Accordingly, in one embodiment, the invention is directed to a method of treating or preventing a microbial infection. The method comprises administering to a vertebrate subject a pharmaceutically effective amount of a composition comprising a porcine β-defensin.

In certain embodiments, the microbial infection is caused by a microbe selected from the group consisting of a bacteria, a virus, a fungus and a parasite, such as a Gram-negative bacterium, for example a Gram-negative bacterium selected from the group consisting of Actinobacillus pleuropneumoniae, Bordetella pertussis Bordetella parapertussis, Streptococcus suis and Escherichia coli.

In additional embodiments, the porcine β-defensin is a porcine β-defensin-1 (pBD-1). In particular embodiments, the pBD-1 comprises residues 23-64 of the contiguous amino acid sequence of SEQ ID NO:2 depicted in FIG. 1B, or an amino acid sequence with at least 75% sequence identity thereto, such as with at least 85% or at least 95% identity to the contiguous amino acid sequence of residues 23-64 of SEQ ID NO:2.

In yet further embodiments, the pBD-1 comprises residues 1-64 of the contiguous amino acid sequence of SEQ ID NO:2 depicted in FIG. 1B, or an amino acid sequence with at least 75% sequence identity thereto, such as with at least 85% or at least 95% identity to the contiguous amino acid sequence of residues 1-64 of SEQ ID NO:2.

In an additional embodiment, the invention is directed to a method of treating or preventing Bordetella pertussis infection. The method comprises administering to a mammalian subject a pharmaceutically effective amount of a composition comprising a porcine β-defensin-1 (pBD-1), wherein the pBD-1 comprises the contiguous amino acid sequence of residues 1-64 of SEQ ID NO:2 depicted in FIG. 1B, or an amino acid sequence with at least 75% sequence identity thereto, such as with at least 85% or at least 95% identity to the contiguous amino acid sequence of residues 1-64 of SEQ ID NO:2 depicted in FIG. 1B.

In yet further embodiments, the invention is directed to a method of enhancing an immunological response to an antigen present in a composition for treating or preventing a microbial infection. The method comprises administering to a vertebrate subject the composition and a pharmaceutically effective amount of a porcine β-defensin.

In certain embodiments, the microbial infection is caused by a microbe selected from the group consisting of a bacteria, a virus, a fungus and a parasite, such as a Gram-negative bacterium, for example a Gram-negative bacterium selected from the group consisting of Actinobacillus pleuropneumoniae, Bordetella pertussis Bordetella parapertussis, Streptococcus suis and Escherichia coli.

In additional embodiments, the porcine β-defensin is a porcine β-defensin-1 (pBD-1). In particular embodiments, the pBD-1 comprises residues 23-64 of the contiguous amino acid sequence of SEQ ID NO:2 depicted in FIG. 1B, or an amino acid sequence with at least 75% sequence identity thereto, such as with at least 85% or at least 95% identity to the contiguous amino acid sequence of residues 23-64 of SEQ ID NO:2 depicted in FIG. 1B.

In yet further embodiments, the pBD-1 comprises residues 1-64 of the contiguous amino acid sequence of SEQ ID NO:2 depicted in FIG. 1B, or an amino acid sequence with at least 75% sequence identity thereto, such as with at least 85% or at least 95% identity to the contiguous amino acid sequence of residues 1-64 of SEQ ID NO:2 depicted in FIG. 1B.

In an additional embodiment, the subject invention is directed to a method of enhancing an immunological response to a Bordetella pertussis antigen. The method comprises administering to a vertebrate subject a vaccine composition comprising the Bordetella pertussis antigen, and administering a pharmaceutically effective amount of a porcine β-defensin-1 (pBD-1), wherein the pBD-1 comprises residues 1-64 of the contiguous amino acid sequence of SEQ ID NO:2 depicted in FIG. 1B, or an amino acid sequence with at least 75% sequence identity thereto, such as at least 85% or at least 95% identity to the contiguous amino acid sequence of residues 1-64 of SEQ ID NO:2 depicted in FIG. 1B.

In yet a further embodiment, the invention is directed to a method of modulating an immunological response to a Gram-negative bacterium, such as a Bordetella pertussis antigen. The method comprises administering to a vertebrate subject a pharmaceutically effective amount of a porcine β-defensin.

In additional embodiments, the porcine β-defensin is a porcine β-defensin-1 (pBD-1). In particular embodiments, the pBD-1 comprises residues 23-64 of the contiguous amino acid sequence of SEQ ID NO:2 depicted in FIG. 1B, or an amino acid sequence with at least 75% sequence identity thereto, such as with at least 85% or at least 95% identity to the contiguous amino acid sequence of SEQ ID NO:2 depicted in FIG. 1B.

In yet further embodiments, the pBD-1 comprises residues 1-64 of the contiguous amino acid sequence of SEQ ID NO:2 depicted in FIG. 1B, or an amino acid sequence with at least 75% sequence identity thereto, such as with at least 85% or at least 95% identity to the contiguous amino acid sequence of residues 1-64 of SEQ ID NO:2 depicted in FIG. 1B.

These and other embodiments of the subject invention will readily occur to those of skill in the art in view of the disclosure herein.

### BRIEF DESCRIPTION OF THE FIGURES

FIGS. 1A and 1B (SEQ ID NOS:1 and 2) show the nucleotide sequence and amino acid sequence, respectively, of prepro pBD-1.

FIG. 2 shows the in vitro inhibitory activity of pBD-1 against B. pertussis.

FIG. 3 shows the in vivo bactericidal activity of pBD-1 against B. pertussis.

FIG. 4 shows the effect of time and dose of pBD-1 on B. pertussis growth.

FIGS. 5A and 5B show the sensitivity of B. pertussis to synthetically derived pBD-1. FIG. 5A shows a bar graph comparing the number of bacteria (CFU/ml) in cultures treated with different concentrations of pBD-1 over time. 5-9×10⁶ CFU B. pertussis were co-cultured with different concentrations of pBD-1 (20 μg/ml, 40 μg/ml and 80 μg/ml) in Stainer-Scholte (SS) medium (control) for 6, 18 and 24 hours. Bacterial numbers were determined by plate counts. FIG. 5B shows a bar graph comparing the number of bacteria (CFU/ml) in cultures in diluted SS medium treated with 10 μg/ml of pBD-1 for 2 or 6 hours. 5-9×10⁶ CFU B. pertussis were co-cultured with 10 μg/ml of pBD-1 in 1/10 diluted SS medium in PBS for 6 or 24 hours and the number of bacteria were determined by plate counts. Five independent experiments were performed.

FIGS. 6A-6D show the in vitro inhibitory activity of pBD-1 against Actinobacillus pleuropneumoniae (6A), Streptococcus suis (6B), B. bronshiseptica (6C) and B. pertussis (6D).

FIGS. 7A and 7B show the susceptibility of B. pertussis and B. bronchiseptica, respectively, to treatment with pBD-1 and hBD-2. FIGS. 7A and 7B show bar graphs comparing the number of bacteria (CFU/ml) in cultures treated with pBD-1 or hBD-2 over time. 5×10⁶ CFU/ml of B. pertussis (FIG. 7A) or B. bronchiseptica (FIG. 7B) were exposed to 40 μg of pBD-1 or 40 μg of hBD-2 for 6 and 24 hr. The number of bacteria was determined by plate counts.

FIG. 8 shows a bar graph comparing the bactericidal effect of bronchoalveolar lavage (BAL) fluid from either newborn piglets or piglets 4-5 weeks old. 5-7×10⁶ CFU B. pertussis were co-cultured for 6 or 24 hours with SS medium (control) or BALs obtained from newborn colostrum-fed, newborn colostrum-deprived, or piglets 4-5 weeks old. Supernatants were plated out onto BG-agar plates to quantify the number of viable bacteria.

FIG. 9 shows the salt dependency of BAL antimicrobial activity. 5-7×10⁶ CFU B. pertussis were co-cultured with SS-medium (control) or BALs obtained from piglets 4-5 weeks old. Various concentrations of NaCl (10 mM, 70 mM, and 100 mM) were added to the SS-medium (control+NaCl) or BALs and incubated for 24 hours. Supernatants were plated onto BG-agar plates to quantify the number of viable bacteria.

FIGS. 10A and 10B show the effect of in vivo treatment with pBD-1 on the bacterial load in the lung. FIG. 10A shows a bar graph comparing the number of bacteria (CFU/ml) in BALs for untreated control and treated piglets over time. At each time point, 3 piglets from the saline treated group (control) and the pBD-1 treated group (Treated) were euthanized. BALs were collected, diluted and plated onto BG agar plates to determine viable bacteria within the BAL. FIG. 10B shows a bar graph comparing the number of bacteria (CFU/ml) in tissues from untreated control and treated piglets over time. Macroscopically, altered tissues were collected, weighed and homogenized. Cleared supernatants were plated onto BG plates to determine bacterial counts.

FIG. 11 shows the tissue expression of pBD-1 mRNA in either newborn piglets (colostrum-fed/colostrum-deprived) or piglets 4-5 weeks old. Tissue samples, collected from animals 4-5 weeks old, newborn colostrums-deprived, or newborn colostrums-fed, were analyzed for a PCR product of 287 base pairs (bp) in length. Controls include water (Neg.) and pBD-1 containing plasmid (Pos). Expression was found only in the tongue of newborn piglets, but found in all investigated tissues of piglets 4-5 weeks old. Six animals per age group were analyzed.

### DETAILED DESCRIPTION OF THE INVENTION

The practice of the present invention will employ, unless otherwise indicated, conventional methods of microbiology, chemistry, biochemistry, recombinant DNA techniques and immunology, within the skill of the art. Such techniques are explained fully in the literature. See, e.g., Handbook of Experimental Immunology, Vols. I-IV (D. M. Weir and C. C. Blackwell eds., Blackwell Scientific Publications); T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

The following amino acid abbreviations are used throughout the text:
Alanine: Ala (A) Arginine: Arg (R)
Asparagine: Asn (N) Aspartic acid: Asp (D)
Cysteine: Cys (C) Glutamine: Gln (Q)
Glutamic acid: Glu (E) Glycine: Gly (G)
Histidine: His (H) Isoleucine: Ile (I)
Leucine: Leu (L) Lysine: Lys (K)
Methionine: Met (M) Phenylalanine: Phe (F)
Proline: Pro (P) Serine: Ser (S)
Threonine: Thr (T) Tryptophan: Trp (W)
Tyrosine: Tyr (Y) Valine: Val (V)

I. Definitions

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a pBD-1” includes a mixture of two or more pBD-1s, and the like.

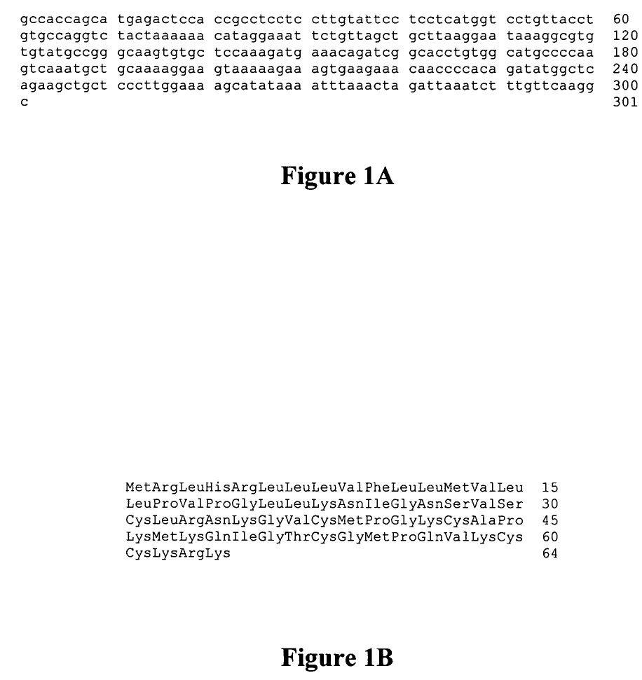

By “porcine β-defensin” is meant any of the various β-defensins of porcine origin. The DNA and corresponding amino acid sequences for various porcine β-defensins, including porcine β-defensin-1 (pBD-1), porcine β-defensin-2, porcine β-defensin-3, porcine β-defensin-4, etc. are known and described in detail below. For example, the nucleotide sequence and corresponding amino acid sequence for a representative porcine β-defensin, pBD-1, are shown in FIGS. 1A-1B, respectively. Porcine β-defensins for use in the present methods include the full-length (i.e., the entire prepro molecule) or substantially full-length proteins, as well as biologically active fragments, fusions or mutants of the proteins. The term also includes postexpression modifications of the polypeptide, for example, glycosylation, acetylation, phosphorylation and the like. Furthermore, for purposes of the present invention, a “porcine β-defensin” refers to a protein which includes modifications, such as deletions, additions and substitutions (generally conservative in nature), to the native sequence, so long as the protein maintains the desired activity. These modifications may be deliberate, as through site-directed mutagenesis, or may be accidental, such as through mutations of hosts which produce the proteins or errors due to PCR amplification. It is readily apparent that the porcine β-defensin may therefore comprise the entire prepro sequence, the mature sequence, fragments, truncated and partial sequences, as well as analogs, muteins and precursor forms of the molecule. The term also intends deletions, additions and substitutions to the reference sequence, so long as the molecule retains the desired biological activity.

The term “derived from” is used to identify the original source of a molecule (e.g., porcine) but is not meant to limit the method by which the molecule is made which can be, for example, by chemical synthesis or recombinant means.

The terms “analog” and “mutein” refer to biologically active derivatives of the reference molecule, that retain desired activity as described herein. In general, the term “analog” refers to compounds having a native polypeptide sequence and structure with one or more amino acid additions, substitutions (generally conservative in nature) and/or deletions, relative to the native molecule, so long as the modifications do not destroy activity and which are “substantially homologous” to the reference molecule as defined below. The term “mutein” refers to peptides having one or more peptide mimics (“peptoids”), such as those described in International Publication No. WO 91/04282. Preferably, the analog or mutein has at least the same desired activity as the native molecule. Methods for making polypeptide analogs and muteins are known in the art and are described further below.

The term also encompasses purposeful mutations that are made to the reference molecule. Particularly preferred analogs include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the molecule of interest may include up to about 5-10 conservative or non-conservative amino acid substitutions, or even up to about 15-20 conservative or non-conservative amino acid substitutions, or any integer between 5-20, so long as the desired function of the molecule remains intact. One of skill in the art can readily determine regions of the molecule of interest that can tolerate change by reference to Hopp/Woods and Kyte-Doolittle plots, well known in the art.

By “fragment” is intended a molecule consisting of only a part of the intact full-length polypeptide sequence and structure. The fragment can include a C-terminal deletion, an N-terminal deletion, and/or an internal deletion of the native polypeptide. A fragment will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, or any integer between 5 amino acids and the full-length sequence, provided that the fragment in question retains the ability to elicit the desired biological response.

By “immunogenic fragment” is meant a fragment of a β-defensin which includes one or more epitopes and thus can modulate an immune response or can act as an adjuvant for a co-administered antigen. Such fragments can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., Proc. Natl. Acad. Sci USA (1981) 78:3824-3828 for determining antigenicity profiles, and the Kyte-Doolittle technique, Kyte et al., J. Mol. Biol. (1982) 157:105-132 for hydropathy plots.

Immunogenic fragments, for purposes of the present invention, will usually be at least about 2 amino acids in length, more preferably about 5 amino acids in length, and most preferably at least about 10 to 15 amino acids in length. There is no critical upper limit to the length of the fragment, which could comprise nearly the full-length of the protein sequence, or even a fusion protein comprising two or more epitopes of the β-defensin in question.

The term “epitope” refers to the site on an antigen or hapten to which specific B cells and T cells respond. The term is also used interchangeably with “antigenic determinant” or “antigenic determinant site.” Antibodies that recognize the same epitope can be identified in a simple immunoassay showing the ability of one antibody to block the binding of another antibody to a target antigen.

An “immunological response” to a composition is the development in the host of a cellular and/or antibody-mediated immune response to the composition or vaccine of interest. Usually, an “immunological response” includes but is not limited to one or more of the following effects: the production of antibodies, B cells, helper T cells, suppressor T cells, and/or cytotoxic T cells and/or γδ T cells, directed specifically to an antigen or antigens included in the composition or vaccine of interest. Preferably, the host will display a protective immunological response to the microorganism in question, e.g., the host will be protected from subsequent infection by the pathogen and such protection will be demonstrated by either a reduction or lack of symptoms normally displayed by an infected host or a quicker recovery time.

The terms “immunogenic” protein or polypeptide refer to an amino acid sequence which elicits an immunological response as described above. An “immunogenic” protein or polypeptide, as used herein, includes the full-length sequence of the β-defensin in question, including the precursor and mature forms, analogs thereof, or immunogenic fragments thereof.

“Substantially purified” generally refers to isolation of a substance (compound, polynucleotide, protein, polypeptide, polypeptide composition) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro-molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

“Homology” refers to the percent identity between two polynucleotide or two polypeptide moieties. Two nucleic acid, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules (the reference sequence and a sequence with unknown % identity to the reference sequence) by aligning the sequences, counting the exact number of matches between the two aligned sequences, dividing by the length of the reference sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The terms “effective amount” or “pharmaceutically effective amount” of a porcine β-defensin or a composition comprising the same, refer to a nontoxic but sufficient amount of the composition to provide the desired response, such as bactericidal activity, enhanced immunogenicity, and, optionally, a corresponding therapeutic effect. The exact amount required will vary from subject to subject, depending on the species, age, and general condition of the subject, the severity of the condition being treated, and the particular porcine β-defensin of interest, mode of administration, and the like. An appropriate “effective” amount in any individual case may be determined by one of ordinary skill in the art using routine experimentation.

By “vertebrate subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The invention described herein is intended for use in any of the above vertebrate species, since the immune systems of all of these vertebrates operate similarly.

The term “treatment” as used herein refers to either (1) the prevention of infection or reinfection (prophylaxis), or (2) the reduction or elimination of symptoms of the disease of interest (therapy).

II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery that porcine β-defensins confer protection against bacterial infection in a reliable animal challenge model. The inventors herein have further shown that porcine beta defensin-1 (pBD-1) displayed antimicrobial activity against a variety of Gram-negative bacteria including E. coli, Actinobacillus pleuropneumoniae, Stretococcus suis and Bordetella pertussis, the causative agent of pertussis or whooping cough in humans. In particular, pBD-1 displayed strong antimicrobial activity in vitro and in vivo and even conferred complete protection against the disease in infected animals. As shown in the examples, following administration of pBD-1, no clinical symptoms or pathological alterations were found in infected animals, whereas untreated infected animals displayed clinical symptoms with severe bronchopneumonia and/or pneumonia. Thus, porcine β-defensins, such as pBD-1, biologically active fragments and analogs thereof, such as molecules with substantial sequence homology thereto, are useful for the prevention and treatment of infectious diseases caused by a variety of infectious microorganisms including diseases caused by bacteria, fungi, parasites and viruses. The porcine β-defensins are particularly useful for the prevention and treatment of pertussis (whooping cough) in humans and other animals.

For example, the porcine β-defensins can be used to treat or prevent a wide variety of infections caused by the various Bordetella species including B. pertussis, B. parapertussis, B. bronhiseptica, and the like; various Neisserial species, including N. meningitidis, N. gonorrhoeae, etc.; various Enterobacteriaceae such as but not limited to Salmonella, such as S. typhimurium, S. enteritidis, Shigella, such as S. flexneri, Escherichia, such as E. coli 0157:H7, Klebsiella, Enterobacter, Serratia, Proteus, Morganella, Providencia, Yersinia, such as Y. enterocolitica, Listeria, such as L. monocytogene, Staphylococcus, such as S. aureus; various Pseudomonas species, such as P. aeruginosa; Stretococcal species, such as S. suis, S. uberis, S. agalactiae, S. dysgalactiae, S. pneumoniae, S. pyogenes, and the like; various Actinobacillus species, including but not limited to A. Pleuropneumoniae, A. suis, A. pyogenes, etc.

Thus, the porcine β-defensins can be used to treat or prevent diseases caused by improper food handling, as well as diseases caused by food-borne pathogens, such as but not limited to Salmonella Enteritidis, Salmonella typhimurium, Escherichia coli 0157:H7, Yersinia enterocolitica, Shigella flexneri, Listeria monocytogene, and Staphylococcus aureus. The porcine β-defensins are also useful against pathogens that cause nosocomial infections, such as but not limited to pathogens that produce extended spectrum β-lactamases (ESBL) and thus have the ability to inactivate β-lactam antibiotics. These enzymes are produced by various bacteria, including Klebsiella pneumoniae, Proteus mirabilis, E. coli and Proteus mirabilis. Additionally, the porcine β-defensin can be used to treat or prevent diseases caused by biocontamination of the skin by pathogenic microorganisms such as Staphylococcus aureus, S. epidermitidis, Pseudomonas aeruginosa, Acinetobacter spp., Klebsiella pneumoniae, Enterobacter cloacae, E. coli, Proteus spp. and fungi such as Candida albicans.

The porcine β-defensins can also be used to treat or prevent respiratory conditions such as caused by Streptococcus pneumoniae, Haemophilus influenzae, and Pseudomonas aeruginosa, as well as sexually transmitted diseases, including but not limited to Chlamydia infections, such as caused by Chlamydia trachomatis and gonococcal infections, such as caused by Neisseria gonorrhoeae.

Additionally, the porcine β-defensins can be used to treat or prevent a number of viral diseases, such as but not limited to those diseases caused by members of the families Picornaviridae (e.g., polioviruses, etc.); Caliciviridae; Togaviridae (e.g., rubella virus, dengue virus, etc.); Flaviviridae; Coronaviridae; Reoviridae; Bimaviridae; Rhabodoviridae (e.g., rabies virus, etc.); Filoviridae; Paramyxoviridae (e.g., mumps virus, measles virus, respiratory syncytial virus, etc.); Orthomyxoviridae (e.g., influenza virus types A, B and C, etc.); Bunyaviridae; Arenaviridae; Retroviradae (e.g., HTLV-I; HTLV-II; HIV-1 (also known as HTLV-III, LAV, ARV, hTLR, etc.). See, e.g. Virology, 3rd Edition (W. K. Joklik ed. 1988); Fundamental Virology, 2nd Edition (B. N. Fields and D. M. Knipe, eds. 1991), for a description of these and other viruses. Other particular examples of viruses include the herpesvirus family of viruses, for example bovine herpes virus (BHV) and human herpes simplex virus (HSV) types 1 and 2, such as BHV-1, BHV-2, HSV-1 and HSV-2, varicella zoster virus (VZV), Epstein-Barr virus (EBV), cytomegalovirus (CMV), HHV6 and HHV7; diseases caused by the various hepatitis viruses, such as HAV, HBV and HCV; diseases caused by papilloma viruses and rotaviruses, etc.

Similarly, the porcine β-defensins will find use against a variety of parasites, such as but not limited to Plasmodium yoelii, P. falciparum, Toxoplasma gondii, Schistosoma japonicum, Leishmania major, Trypanosoma cruzi, and so forth. It is readily apparent that the subject invention can be used to prevent or treat a wide variety of diseases.

Additionally, the porcine β-defensins find use against a number of fungal pathogens, such but not limited to those fungi causing Candidiasis, Cryptococcosis, Asperigillosis, Zygomycosis, Blastomycosis, Coccidioidomycosis, Histoplasmosis, Paracoccidiodomycosis, Sporotrichosis.

Moreover, porcine β-defensins are useful as adjuvants to be provided in combination with vaccines, in order to enhance an immune response, such as a cell-mediated or humoral immune response, to the co-delivered antigen. For example, porcine β-defensins, such as pBD-1, biologically active fragments and analogs thereof, such as molecules with substantial sequence homology thereto, can be co-administered with commercially available animal and human vaccines, including but not limited to pertussis vaccines and combination vaccines, such as the various whole cell (wP) and acellular vaccines (aP). Nonlimiting examples of such vaccines include the vaccines known as TRIPEDIA, TRIPACEL, QUADRACEL, TETRAVAL, TETRACT-Hib, PENTACT-Hib, PENTACEL, PENTAVAC, and HEXAVAC (Aventis, Bridgewater, N.J.); INFANRIX and PEDIARIX (GlaxoSmithKline, Research Triangle Park, N.C.); CERTIVA (North American Vaccine, Beltsville, Md.); BIOTHRAX; TICE BCG; MYCOBAX; HiBTITER; PEDVAXHIB; ACTHIB; COMVAX; HAVRIX; VAQTA; TWINRIX; RECOMBIVAX HB; ENGERIX-B; FLUMIST; FLUVIDRIN; FLUZONE; JE-VAX; ATTENUVAX; M-M-VAX; M-M-R II; MENUMONE-A/C/Y/W-135; MUMPSVAX; PNEUMOVAX 23; PREVNAR; POLIOVAX; IPOL; IMOVAX; RABAVERT; MERUVAX II; DRYVAX; TYPHIM Vi; VIVOTIF; VARIVAX; YF-VAX.

The β-defensins can be administered prior to, concurrently with, or subsequent to the vaccine composition. If administered concurrently, the porcine β-defensins can be administered in the same or in a different composition. If provided in a different composition, the β-defensin can be administered at the same or different site of administration.

Furthermore, the porcine β-defensins can be used to modulate the immune response against a variety of microorganisms, including but not limited to bacteria, fungi, parasites and viruses. In a particularly preferred embodiment, the porcine β-defensins are used to modulate the immune response against Gram-negative bacteria, such as but not limited to pertussis, in humans and other animals. For example, porcine β-defensins, such as pBD-1, biologically active fragments and analogs thereof, such as molecules with substantial sequence homology thereto, can be used to stimulate chemotaxis of immature dendritic cells and T-cells, glucocorticoid production, macrophage phagocytosis, mast cell degranulation, complement activation, IL-8 production by epithelial cells and prostaglandin D(2) production.

Additionally, the porcine β-defensins can be used in combination with an antibacterial agent. Such agents include, without limitation, penicillins, cephalosporins, carbacephems, cephamycins, erythromycins, carbapenems, monobactams, aminoglycosides, glycopeptides, quinolones, tetracyclines, macrolides, and fluoroquinolones, such as Penicillin G (CAS Registry No.: 61-33-6); Methicillin (CAS Registry No.: 61-32-5); Nafcillin (CAS Registry No.: 147-52-4); Oxacillin (CAS Registry No.: 66-79-5); Cloxacillin (CAS Registry No.: 61-72-3); Dicloxacillin (CAS Registry No.: 3116-76-5); Ampicillin (CAS Registry No.: 69-53-4); Amoxicillin (CAS Registry No.: 26787-78-0); Ticarcillin (CAS Registry No.: 34787-01-4); Carbenicillin (CAS Registry No.: 4697-36-3); Mezlocillin (CAS Registry No.: 51481-65-3); Azlocillin (CAS Registry No.: 37091-66-0); Piperacillin (CAS Registry No.: 61477-96-1); Imipenem (CAS Registry No.: 74431-23-5); Aztreonam (CAS Registry No.: 78110-38-0); Cephalothin (CAS Registry No.: 153-61-7); Cefazolin (CAS Registry No.: 25953-19-9); Cefaclor (CAS Registry No.: 70356-03-5); Cefamandole formate sodium (CAS Registry No.: 42540-40-9); Cefoxitin (CAS Registry No.: 35607-66-0); Cefuroxime (CAS Registry No.: 55268-75-2); Cefonicid (CAS Registry No.: 61270-58-4); Cefinetazole (CAS Registry No.: 56796-20-4); Cefotetan (CAS Registry No.: 69712-56-7); Cefprozil (CAS Registry No.: 92665-29-7); Loracarbef (CAS Registry No.: 121961-22-6); Cefetamet (CAS Registry No.: 65052-63-3); Cefoperazone (CAS Registry No.: 62893-19-0); Cefotaxime (CAS Registry No.: 63527-52-6); Ceftizoxime (CAS Registry No.: 68401-81-0); Ceftriaxone (CAS Registry No.: 73384-59-5); Ceftazidime (CAS Registry No.: 72558-82-8); Cefepime (CAS Registry No.: 88040-23-7); Cefixime (CAS Registry No.: 79350-37-1); Cefpodoxime (CAS Registry No.: 80210-62-4); Cefsulodin (CAS Registry No.: 62587-73-9); Fleroxacin (CAS Registry No.: 79660-72-3); Nalidixic acid (CAS Registry No.: 389-08-2); Norfloxacin (CAS Registry No.: 70458-96-7); Ciprofloxacin (CAS Registry No.: 85721-33-1); Ofloxacin (CAS Registry No.: 82419-36-1); Enoxacin (CAS Registry No.: 74011-58-8); Lomefloxacin (CAS Registry No.: 98079-51-7); Cinoxacin (CAS Registry No.: 28657-80-9); Doxycycline (CAS Registry No.: 564-25-0); Minocycline (CAS Registry No.: 10118-90-8); Tetracycline (CAS Registry No.: 60-54-8); Amikacin (CAS Registry No.: 37517-28-5); Gentamicin (CAS Registry No.: 1403-66-3); Kanamycin (CAS Registry No.: 8063-07-8); Netilmicin (CAS Registry No.: 56391-56-1); Tobramycin (CAS Registry No.: 32986-56-4); Streptomycin (CAS Registry No.: 57-92-1); Azithromycin (CAS Registry No.: 83905-01-5); Clarithromycin (CAS Registry No.: 81103-11-9); Erythromycin (CAS Registry No.: 114-07-8); Erythromycin estolate (CAS Registry No.: 3521-62-8); Erythromycin ethyl succinate (CAS Registry No.: 41342-53-4); Erythromycin glucoheptonate (CAS Registry No.: 23067-13-2); Erythromycin lactobionate (CAS Registry No.: 3847-29-8); Erythromycin stearate (CAS Registry No.: 643-22-1); Vancomycin (CAS Registry No.: 1404-90-6); Teicoplanin (CAS Registry No.: 61036-64-4); Chloramphenicol (CAS Registry No.: 56-75-7); Clindamycin (CAS Registry No.: 18323-44-9); Trimethoprim (CAS Registry No.: 738-70-5); Sulfamethoxazole (CAS Registry No.: 723-46-6); Nitrofurantoin (CAS Registry No.: 67-20-9); Rifampin (CAS Registry No.: 13292-46-1); Mupirocin (CAS Registry No.: 12650-69-0); Metronidazole (CAS Registry No.: 443-48-1); Cephalexin (CAS Registry No.: 15686-71-2); Roxithromycin (CAS Registry No.: 80214-83-1); Co-amoxiclavuanate; combinations of Piperacillin and Tazobactam; and their various salts, acids, bases, and other derivatives.

In order to further an understanding of the invention, a more detailed discussion is provided below regarding porcine β-defensins and compositions for use in the subject methods.

Porcine β-Defensins

Porcine β-defensins for use in any of the above methods include any member of the porcine β-defensin family, including without limitation porcine β-defensin-1, porcine β-defensin-2, porcine β-defensin-3, porcine β-defensin-4, and so on. Sequences for these molecules are known. For example, the sequence for a representative porcine β-defensin, porcine β-defensin-1 (pBD-1), is shown in FIGS. 1A-1B herein and is also described in NCBI accession numbers AF031666 and NM_{—}213838; the sequence for porcine β-defensin-2 is described in NCBI accession number AY06573; the sequence for porcine β-defensin-3 is described in AY460575; the sequence for porcine β-defensin-4 is described in NCBI accession number AY460576. Any of these sequences, as well as fragments and analogs thereof, that display the appropriate biological activity, such as bactericidal activity in vitro, therapeutic or prophylactic activity in vivo, or the ability to modulate an immune response, such as to enhance an immune response to a co-delivered antigen, will find use in the present methods.

For example, porcine β-defensins for use herein can include the entire prepro sequence, the pro-protein without the pre sequence, or the mature protein without the prepro sequence. By way of example, pBD-1 (SEQ ID NO:2), depicted in FIG. 1B herein, includes a signal sequence at amino acid positions 1-20. The prepro sequence spans amino acid residues 1-64 of FIG. 1B. The pro sequence spans amino acid residues 21-64 and the mature protein includes amino acid residues 23-64 of FIG. 1B. It is readily apparent that a porcine β-defensin for use herein can take any number of forms, so long as the molecule retains the desired biological activity. Thus, a pBD-1 molecule for use herein can include, but need not include, the native signal sequence, along with the pro-sequence or the mature sequence. Alternatively, a pBD-1 for use herein can include the pro sequence or mature sequence with a heterologous signal sequence. Alternatively, a β-defensin for use herein can include only the sequence of the mature protein, so long as the molecule retains biological activity. Moreover, β-defensins for use herein can be biologically active molecules that display substantial homology to the parent molecule, as defined above.

Thus, β-defensins for use with the present invention can include, for example, the entire parent molecule, or biologically active fragments thereof, such as fragments including contiguous amino acid sequences comprising at least about 5-10 up to about 50 to the full-length of the molecule in question, or any integer therebetween. Typically, such fragments will at least include the β-defensin consensus sequence which contains six invariantly spaced cysteine residues. This sequence is found at amino acid positions 31-61 of the pBD-1 depicted in FIG. 1B. If the porcine β-defensin is to be used as an adjuvant or to modulate an immune response, the molecule will typically include one or more epitopes. Such epitopes are readily identifiable using techniques well known in the art, such as using standard antigenicity and hydropathy plots, for example those calculated using, e.g., the Omiga version 1.0 software program available from the Oxford Molecular Group. This computer program employs the Hopp/Woods method, Hopp et al., Proc. Natl. Acad. Sci USA (1981) 78:3824-3828 for determining antigenicity profiles, and the Kyte-Doolittle technique, Kyte et al., J. Mol. Biol. (1982) 157:105-132 for hydropathy plots. This program can be used with the following parameters: averaging results over a window of 7; determining surface probability according to Emini; chain flexibility according to Karplus-Schulz; antigenicity index according to Jameson-Wolf; secondary structure according to Garnier-Osguthorpe-Robson; secondary structure according to Chou-Fasman; and identifying predicted glycosylation sites. One of skill in the art can readily use the information obtained in combination with teachings of the present specification to identify antigenic regions which should be included in the molecules for use with the present invention.

As explained above, porcine β-defensins, biologically active fragments and analogs thereof will find use in the present methods. Methods for determining biological activity are readily known. For example, the ability of a porcine β-defensin to inhibit bacterial growth in vitro and in vivo can be determined using the techniques described in the examples herein. For in vitro activity, the porcine β-defensin in question can be added to an appropriate medium, with the desired microorganism and incubated for an appropriate time period, such as for 2 hours to 5 days, preferably 6-30 hours. The culture can then be inspected for microbial growth using a UV spectrophotometer or by viability testing using, for example, BG agar plates. Alternatively, the culture can be inspected visually or microscopically for microbial growth. A minimum inhibitory concentration (MIC) of a porcine β-defensin is the lowest concentration of peptide that completely inhibits growth of the organism. Molecules that exhibit good activity against the test strain, typically having an MIC of less than or equal to 50 μg/ml, are selected for further testing. Alternatively, time kill curves can be used to determine the differences in colony counts over a set time period, typically 24 hours or more. Briefly, a suspension of organisms of known concentration is prepared and a porcine β-defensin is added. Aliquots of the suspension are removed at set times, diluted, plated on medium, incubated, and counted. MIC is measured as the lowest concentration of the β-defensin that completely inhibits growth of the organism. In general, lower MIC values are preferred.

Similarly, a porcine β-defensin molecule of interest may be assessed in vivo for its ability to ameliorate microbial infections using animal models. A variety of methods and animal models are available for assessment of antibacterial activity, such as acute infection models including those in which (a) normal animals, such as mice, receive a lethal dose of microorganisms, (b) neutropenic mice receive a lethal dose of microorganisms or (c) chronic infection models. One especially useful animal model for pertussis is the porcine challenge model described in the examples herein. Generally, a porcine β-defensin is useful as a therapeutic if inhibition of microorganismal growth compared to inhibition with vehicle alone is statistically significant. This measurement can be made directly from cultures isolated from body fluids or sites, or indirectly, by assessing clinical symptoms, organ damage, survival rates and the like, of infected animals.

If the porcine β-defensin is to be used to enhance an immune response to a co-delivered antigen, enhanced immunogenic activity can be determined by determining whether the composition of interest when co-delivered with the β-defensin of interest, possesses a greater capacity to elicit an immune response than the immune response elicited by an equivalent amount of the composition delivered without the co-administered β-defensin. Such enhanced immunogenicity can be determined by administering the composition of interest with and without co-administration of the β-defensin, and comparing antibody titers or cellular immune response produced by the two using standard assays such as radioimmunoassay, ELISAs, lymphoproliferation assays, and the like, well known in the art.

The β-defensins for use with the present invention can be obtained using standard techniques. For example, since the porcine β-defensins are relatively small, they can be conveniently synthesized chemically, by any of several techniques that are known to those skilled in the peptide art. In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like. Typical solid supports are cross-linked polymeric supports. These can include divinylbenzene cross-linked-styrene-based polymers, for example, divinylbenzene-hydroxymethylstyrene copolymers, divinylbenzene-chloromethylstyrene copolymers and divinylbenzene-benzhydrylaminopolystyrene copolymers.

The β-defensins of the present invention can also be chemically prepared by other methods such as by the method of simultaneous multiple peptide synthesis. See, e.g., Houghten Proc. Natl. Acad. Sci. USA (1985) 82:5131-5135; U.S. Pat. No. 4,631,211.

Alternatively, the β-defensins can be produced by recombinant techniques. See, e.g., Zhang et al., FEBS Lett. (1998) 424:37-40; Zhang et al., J. Biol. Chem. (1999) 274:24031-24037; Shi et al., Infect. Immun. (1999) 67:3121-3127 for descriptions of the recombinant production of pBD-1. The porcine β-defensin can be produced recombinantly, e.g., by obtaining a DNA molecule from a cDNA library or vector including the same, or from host tissue using phenol extraction. Alternatively, DNA encoding the desired protein can be synthesized, along with an ATG initiation codon. The nucleotide sequence can be designed with the appropriate codons for the particular amino acid sequence desired. In general, one selects preferred codons for the intended host in which the sequence is expressed. The complete sequence is generally assembled from overlapping oligonucleotides prepared by standard methods and assembled into a complete coding sequence. See, e.g., Edge Nature (1981) 292:756; Nambair et al. Science (1984) 223:1299; Jay et al. J. Biol. Chem. (1984) 259:6311. Automated synthetic techniques such as phosphoramide solid-phase synthesis, can be used to generate the nucleotide sequence. See, e.g., Beaucage, S. L. et al. Tet. Lett. (1981) 22:1859-1862; Matteucci, M. D. et al. J. Am. Chem. Soc. (1981) 103:3185-3191. Next the DNA is cloned into an appropriate vector, either procaryotic or eucaryotic, using conventional methods. Numerous cloning vectors are known to those of skill in the art, and the selection of an appropriate cloning vector is a matter of choice. Suitable vectors include, but are not limited to, plasmids, phages, transposons, cosmids, chromosomes or viruses which are capable of replication when associated with the proper control elements. The coding sequence is then placed under the control of suitable control elements, depending on the system to be used for expression. Thus, the coding sequence can be placed under the control of a promoter, ribosome binding site (for bacterial expression) and, optionally, an operator, so that the DNA sequence of interest is transcribed into RNA by a suitable transformant. The coding sequence may or may not contain a signal peptide or leader sequence which can later be removed by the host in post-translational processing. See, e.g., U.S. Pat. Nos. 4,431,739; 4,425,437; 4,338,397. If present, the signal sequence can be the native leader found in association with the β-defensin of interest.

In addition to control sequences, it may be desirable to add regulatory sequences which allow for regulation of the expression of the sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector. For example, enhancer elements may be used herein to increase expression levels of the constructs. Examples include the SV40 early gene enhancer (Dijkema et al. (1985) EMBO J. 4:761), the enhancer/promoter derived from the long terminal repeat (LTR) of the Rous Sarcoma Virus (Gorman et al. (1982) Proc. Natl. Acad. Sci. USA 79:6777) and elements derived from human CMV (Boshart et al. (1985) Cell 41:521), such as elements included in the CMV intron A sequence (U.S. Pat. No. 5,688,688). The expression cassette may further include an origin of replication for autonomous replication in a suitable host cell, one or more selectable markers, one or more restriction sites, a potential for high copy number and a strong promoter.

An expression vector is constructed so that the particular coding sequence is located in the vector with the appropriate regulatory sequences, the positioning and orientation of the coding sequence with respect to the control sequences being such that the coding sequence is transcribed under the “control” of the control sequences (i.e., RNA polymerase which binds to the DNA molecule at the control sequences transcribes the coding sequence). Modification of the sequences encoding the molecule of interest may be desirable to achieve this end. For example, in some cases it may be necessary to modify the sequence so that it can be attached to the control sequences in the appropriate orientation; i.e., to maintain the reading frame. The control sequences and other regulatory sequences may be ligated to the coding sequence prior to insertion into a vector. Alternatively, the coding sequence can be cloned directly into an expression vector which already contains the control sequences and an appropriate restriction site.

As explained above, it may also be desirable to produce mutants or analogs of the polypeptide of interest. Mutants or analogs of β-defensins for use in the subject compositions may be prepared by the deletion of a portion of the sequence encoding the molecule of interest, by insertion of a sequence, and/or by substitution of one or more nucleotides within the sequence. Techniques for modifying nucleotide sequences, such as site-directed mutagenesis, and the like, are well known to those skilled in the art. See, e.g., Sambrook et al., supra; Kunkel, T. A. (1985) Proc. Natl. Acad. Sci. USA (1985) 82:448; Geisselsoder et al. (1987) BioTechniques 5:786; Zoller and Smith (1983) Methods Enzymol. 100:468; Dalbie-McFarland et al. (1982) Proc. Natl. Acad. Sci USA 79:6409.

The molecules can be expressed in a wide variety of systems, including insect, mammalian, bacterial, viral and yeast expression systems, all well known in the art. For example, insect cell expression systems, such as baculovirus systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit). Similarly, bacterial and mammalian cell expression systems are well known in the art and described in, e.g., Sambrook et al., supra. Yeast expression systems are also known in the art and described in, e.g., Yeast Genetic Engineering (Barr et al., eds., 1989) Butterworths, London.

A number of appropriate host cells for use with the above systems are also known. For example, mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human embryonic kidney cells, human hepatocellular carcinoma cells (e.g., Hep G2), Madin-Darby bovine kidney (“MDBK”) cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Nucleic acid molecules comprising nucleotide sequences of interest can be stably integrated into a host cell genome or maintained on a stable episomal element in a suitable host cell using various gene delivery techniques well known in the art. See, e.g., U.S. Pat. No. 5,399,346.

Depending on the expression system and host selected, the molecules are produced by growing host cells transformed by an expression vector described above under conditions whereby the protein is expressed. The expressed protein is then isolated from the host cells and purified. If the expression system secretes the protein into growth media, the product can be purified directly from the media. If it is not secreted, it can be isolated from cell lysates. The selection of the appropriate growth conditions and recovery methods are within the skill of the art.

The porcine β-defensins, whether produced recombinantly or synthetically, are formulated into compositions and used in methods as detailed above.

β-Defensin Formulations and Administration

The porcine β-defensins can be formulated into compositions, either alone or in combination with antigens, as described above, for delivery to subjects for either inhibiting infection, or for enhancing an immune response to a co-administered antigen or combination of antigens, such as with a combination pertussis vaccine. Methods of preparing such formulations are described in, e.g., Remington's Pharmaceutical Sciences, Mack Publishing Company, Easton, Pa., 18 Edition, 1990. The compositions of the present invention can be prepared as injectables, either as liquid solutions or suspensions. Solid forms suitable for solution in or suspension in liquid vehicles prior to injection may also be prepared. The preparation may also be emulsified or the active ingredient encapsulated in liposome vehicles. The active immunogenic ingredient is generally mixed with a compatible pharmaceutical vehicle, such as, for example, water, saline, dextrose, glycerol, ethanol, or the like, and combinations thereof. In addition, if desired, the vehicle may contain minor amounts of auxiliary substances such as wetting or emulsifying agents and pH buffering agents.

If used to modulate an immune response, additional adjuvants which enhance the effectiveness of the composition may also be added to the formulation. Adjuvants may include for example, muramyl dipeptides, pyridine, aluminum hydroxide, dimethyldioctadecyl ammonium bromide (DDA), oils, oil-in-water emulsions, MF-59, CpG DNA, saponins, cytokines, and other substances known in the art.

The β-defensin may also be linked to a carrier in order to increase the immunogenicity thereof. Suitable carriers include large, slowly metabolized macro-molecules such as proteins, including serum albumins, keyhole limpet hemocyanin, immunoglobulin molecules, thyroglobulin, ovalbumin, and other proteins well known to those skilled in the art; polysaccharides, such as sepharose, agarose, cellulose, cellulose beads and the like; polymeric amino acids such as polyglutamic acid, polylysine, and the like; amino acid copolymers; and inactive virus particles.

The β-defensins may be used in their native form or their functional group content may be modified by, for example, succinylation of lysine residues or reaction with Cys-thiolactone. A sulfhydryl group may also be incorporated by, for example, reaction of amino functions with 2-iminothiolane or the N-hydroxysuccinimide ester of 3-(4-dithiopyridyl propionate. Suitable carriers may also be modified to incorporate spacer arms (such as hexamethylene diamine or other bifunctional molecules of similar size) for attachment of peptides. Other suitable carriers include VP6 polypeptides of rotaviruses, or functional fragments thereof, as disclosed in U.S. Pat. No. 5,071,651, incorporated herein by reference. Also useful is a fusion product of a viral protein and the subject immunogens made by methods disclosed in U.S. Pat. No. 4,722,840. Still other suitable carriers include cells, such as lymphocytes, since presentation in this form mimics the natural mode of presentation in the subject, which gives rise to the immunized state. Methods of coupling peptides to proteins or cells are known to those of skill in the art.

Furthermore, the β-defensins (or complexes thereof) may be formulated into compositions in either neutral or salt forms. Pharmaceutically acceptable salts include the acid addition salts (formed with the free amino groups of the active polypeptides) and which are formed with inorganic acids such as, for example, hydrochloric or phosphoric acids, or such organic acids as acetic, oxalic, tartaric, mandelic, and the like. Salts formed from free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Injectable formulations will contain a “pharmaceutically effective amount” of the active ingredient, that is, an amount capable of achieving the desired response in a subject to which the composition is administered. In the treatment and prevention of pertussis, for example, a “pharmaceutically effective amount” would preferably be an amount which reduces or ameliorates the symptoms of whooping cough. The exact amount is readily determined by one skilled in the art using standard tests. The β-defensin will typically range from about 1% to about 95% (w/w) of the composition, or even higher or lower if appropriate. With the present formulations, 1 μg to 2 mg, such as 100 μg to 1 mg, of active ingredient per ml of injected solution should be adequate to treat or prevent infection when a dose of 1 to 3 ml per animal is administered. If the β-defensin is to be used as an adjuvant, to enhance the immune response to a co-delivered antigen or combination of antigens, the amount delivered will generally be in the range of 2 ng to 5 mg, more generally 5 ng to 500 ng, for example 10 ng to 250 ng, or any amount within these stated ranges. The quantity to be administered depends on the animal to be treated, the capacity of the animal's immune system to synthesize antibodies, and the degree of protection desired. Effective dosages can be readily established by one of ordinary skill in the art through routine trials establishing dose response curves.

The composition can be administered parenterally, e.g., by intratracheal, intramuscular, subcutaneous, intraperitoneal, intravenous injection, or by delivery directly to the lungs. The subject is administered at least one dose of the composition. Moreover, the animal may be administered as many doses as is required to bring about the desired biological effect.

Additional formulations which are suitable for other modes of administration include suppositories and, in some cases, aerosol, intranasal, oral formulations, and sustained release formulations. For suppositories, the vehicle composition will include traditional binders and carriers, such as, polyalkaline glycols, or triglycerides. Such suppositories may be formed from mixtures containing the active ingredient in the range of about 0.5% to about 10% (w/w), preferably about 1% to about 2%. Oral vehicles include such normally employed excipients as, for example, pharmaceutical grades of mannitol, lactose, starch, magnesium, stearate, sodium saccharin cellulose, magnesium carbonate, and the like. These oral vaccine compositions may be taken in the form of solutions, suspensions, tablets, pills, capsules, sustained release formulations, or powders, and contain from about 10% to about 95% of the active ingredient, preferably about 25% to about 70%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Controlled or sustained release formulations are made by incorporating the protein into carriers or vehicles such as liposomes, nonresorbable impermeable polymers such as ethylenevinyl acetate copolymers and HYTREL copolymers, swellable polymers such as hydrogels, resorbable polymers such as collagen and certain polyacids or polyesters such as those used to make resorbable sutures, polyphosphazenes, alginate, microparticles, gelatin nanospheres, chitosan nanoparticles, and the like. The β-defensins can also be delivered using implanted mini-pumps, well known in the art.

III. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Materials and Methods

Bacterial Preparation:

The B. pertussis strain used in these experiments was Tohama I. Bacterial suspensions were stored at −70° C. in casamino acid plus 10% glycerol. Organisms were initially grown on the surface of Bordet-Gengou (BG) agar containing 15% (vol/vol) defibrinated sheep blood and 40 μg/ml of Cephalexin at 37° C. for 48 hrs. The bacterial cells were harvested and washed in phosphate-buffered saline (PBS, pH 7.2) by centrifugation at 2500 g for 10 min. The pellets were resuspended in saline and adjusted to a specified bacterial titer by determining the optical density at 600 nm using a spectrophotometer; The corresponding viable counts of these suspensions were measured by plating out serial dilutions of the bacterial suspension onto BG-agar and incubating at 37° C. for 4-5 days.

E. coli, Actinobacillus pleuropneumoniae and B. bronchiseptica were grown on agar plates using routine procedures. The bacteria were washed off the plates, washed in saline and subsequently resuspended in media. They were co-incubated with pBD-1 in varying doses and incubated for various time periods (6-48 hours). The bacteria were quantified by plating the suspension onto agar plates.

Animals:

Pregnant Landrace sows were purchased from the Saskatoon Prairie Swine Centre, University of Saskatchewan. Sows were induced to farrow by intramuscular (i.m.) injection of prostaglandin (Planate) (Schering, Quebec, Canada) at day 113 of gestation. Piglets were born at day 114-115 of gestation. Nursing piglets were kept within the same room, but in separated pens and monitored very closely. The piglets were challenged at 3-5 days of age. All experiments were conducted in accordance with the ethical guidelines of the University of Saskatchewan and the Canadian Council for Animal Care (CCAC).

Synthesis of the pBD-1 and hBD-2:

Both pBD-1 and hBD-2 were chemically synthesized on a Pioneer solid phase peptide synthesizer (PerSeptive Biosystems. Foster City, Calif., USA) using Fmoc [9-Fluorenylmethyloxycarbonyl] chemistry. The peptide chain was synthesized from the carboxyl terminal to the amino terminal onto PAL-PEG-PS resin. The Fmoc protecting group at the amino terminal was deprotected with piperidine. The peptide was cleaved from the resin with concurrent deprotection of the side chain protecting groups by treating the resin bound peptide with trifluoroacetic acid (TFA, 9.3 parts) in the presence of scavengers: anisole: ethylmethyl sulfide: and 1,2-ethanedithiol (3:3:1 parts) for 7 hours. The crude peptide was filtered from the resin and the TFA evaporated. Diethyl ether was added to the residue to precipitate the crude peptide. The peptide was isolated and purified by high performance liquid chromatography on Vydac Protein C-4 columns (1.0×25 cm) eluting with a linear gradient of 35% A (H₂O, 0.1% TFA)—90% B (ACN/H₂O: 90/10, 0.01% TFA) in 40 min at a flow rate of 3 mL/minute. The purity and molecular weight of the peptide was confirmed by MALDITOF mass spectrometry on a PE Biosystems Voyager System 4068 (National Research Council, Plant Biotechnology Institute, Saskatoon, Canada) and amino acid analysis.

Respiratory Infection and Post Mortem Analysis:

Newborn piglets were anaesthetized with isoflurane and intubated using a laryngoscope. Infection was initiated by delivering 5×10⁹ CFU of B. pertussis strain Tohama I intrapulmonarily (craniodorsal of the bifurcation) through a MICRO-RENATHANE tube (0.95) (Braintree Scientific Inc., Braintree, Mass., USA), which was inserted through an endotracheal tube (3 mm) (Jorgensen Laboratories Inc. Loveland, Colo., USA). The bottom-end of the MICRO-RENATHANE tube was sealed and minute holes were made for the equal distribution of bacteria. Piglets were monitored twice daily for clinical symptoms including fever and respiratory symptoms such as nasal discharge, non paroxysmal cough and breathing difficulties. Piglets were euthanized by i.p. injection of 5 ml of Euthanyl (Sodium barbiturate; Bimeda-MTC, Ont, Canada) at different time points over a 10 day period post challenge. The thoracic and the abdominal cavities and the lungs were examined for any lesions and abnormalities such as pleuritis or local collections of blood and fluids in the thorax were noted.

### EXAMPLE 1

### Bactericidal Activity of pBD-1 In Vitro

A. Bactericidal Activity of Defensin against B. pertussis

To examine the bactericidal effect of defensin against B. pertussis, 10 μl of bacterial suspension (OD_{600 nm} of 0.2) was added to 200 μl of Stainer and Scholte (SS) broth medium with or without chemically synthesized defensin at different concentrations in a 96 well plate and incubated at 37° C. for up to 30 hours. At the end of each incubation period (6 hrs, 18 hrs, 24 hrs, 30 hrs), the culture medium was immediately removed for quantification of viable bacteria. The culture medium with or without defensin was plated onto BG agar plates and incubated at 37° C. for up to 4 days after which time the number of colonies were counted. The number of surviving bacteria was estimated by colony-forming units (CFU). The results were confirmed by three independent experiments compared with results from control bacterial culture (no peptide).

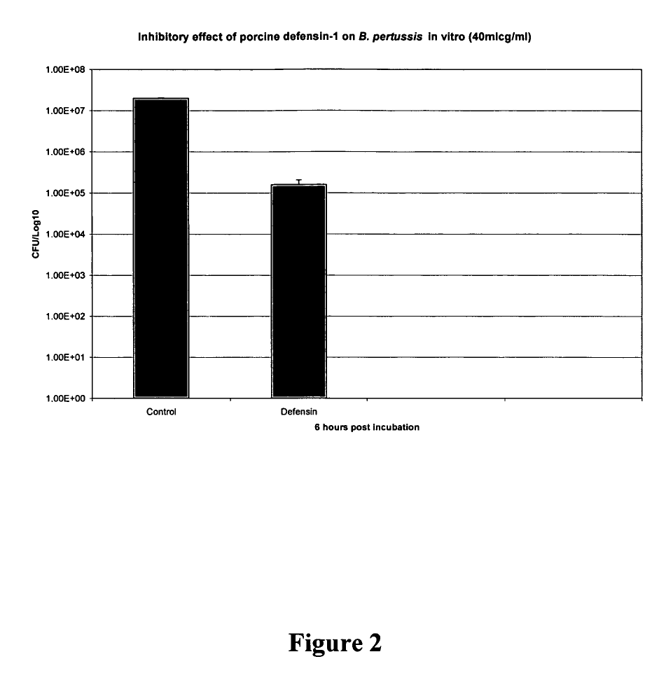

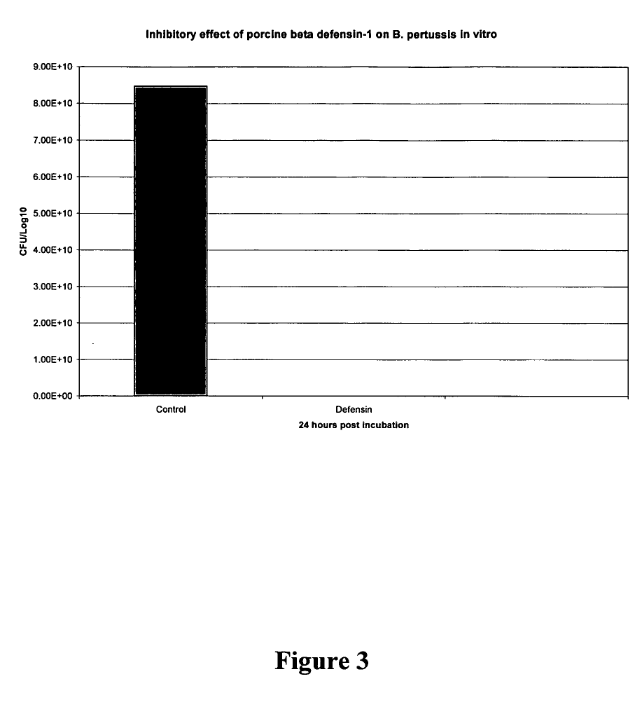

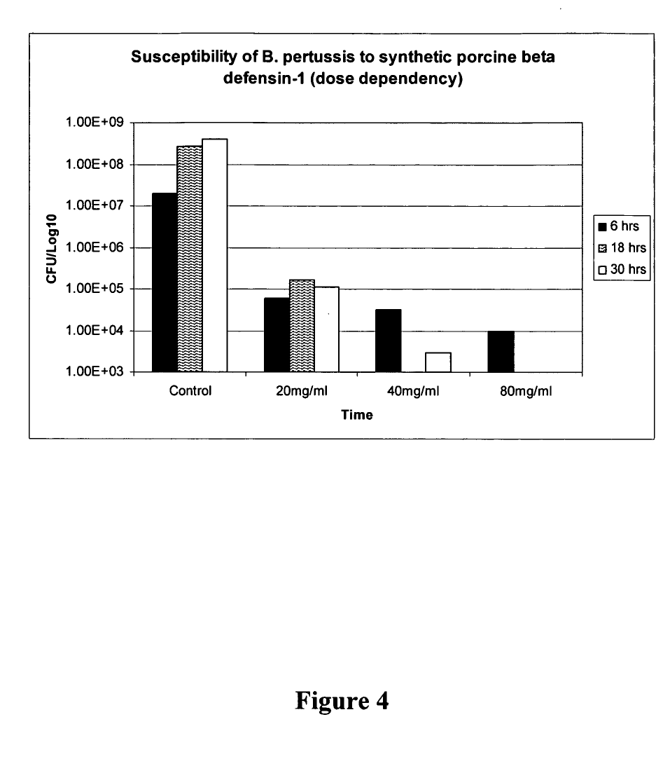

FIG. 2 shows the susceptibility of B. pertussis to 40 μg of pBD-1 following 6 hours of incubation and incubated for 4 days on BG plates at 37° C. FIG. 3 shows the susceptibility of B. pertussis to 40 μg of pBD-1 after 24 hours of incubation and then plated in serial 2-fold dilution on BG plates and incubated for 4 days plates at 37° C. FIG. 4 shows the dose dependent-inhibitory effect of pBD-1 on B. pertussis at different incubation periods in vitro. 20, 40 and 80 μg of pBD-1 was added to bacterial culture and incubated at 37° C. Aliquots were removed at 6, 18 and 30 hr post incubation and incubated for 4 days on BG plates at 37° C.

As shown in the figures, the inhibitory effect of pBD-1 against B. pertussis was dose and time dependent. Growth reduction was seen after 6 hours of incubation. Wells that were co-cultured with pBD-1 displayed about 2 logs reduction in bacterial growth compared to untreated control wells. After 18 hours, the pBD-1 reduced the bacterial growth by more than 100,000-fold, depending on the dose used. After 30 hours, a 1,000,000-fold reduction of the bacterial growth was observed. During these incubation periods, the viability of the control bacteria remained at or above 99%. As shown in FIG. 4, inhibitory activity of the pBD-1 was highest at 80 μg/ml, but complete inhibition was also found at 40 μg/ml at 18 hours of incubation.

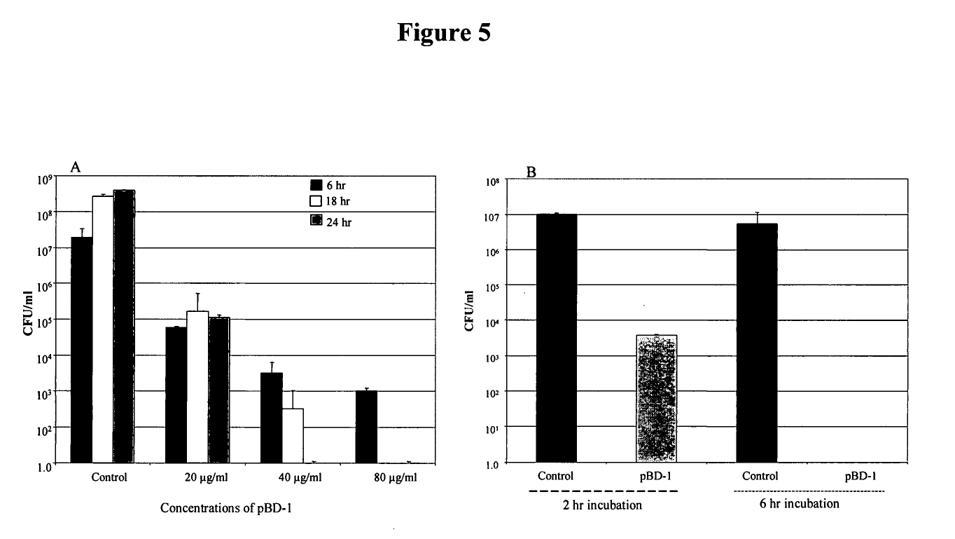

Assays of the inhibitory effect of pBD-1 against B. pertussis were repeated. The inhibitory effect of pBD-1 against B. pertussis was tested with inhibition assays using different concentrations of chemically synthesized pBD-1 (20, 40 and 80 μg/ml). As shown in FIG. 5A, at concentrations of 20 μg/ml of pBD-1 the growth of B. pertussis was inhibited by at least 2 logs after 6 hours of incubation in comparison with control medium. Higher concentrations of pBD-1 and prolonged incubation resulted in significantly increased inhibitory activity. (FIG. 5A). Indeed, at 80 μg/ml of pBD-1 and after 18 hours of incubation, 5-8×10⁶ CFU of B. pertussis were completely neutralized. These results demonstrate that chemically synthesized pBD-1 displays strong bactericidal activity against B. pertussis in vitro in both a time and dose-dependent manner.

B. Bactericidal Activity of Defensin against Other Bacteria

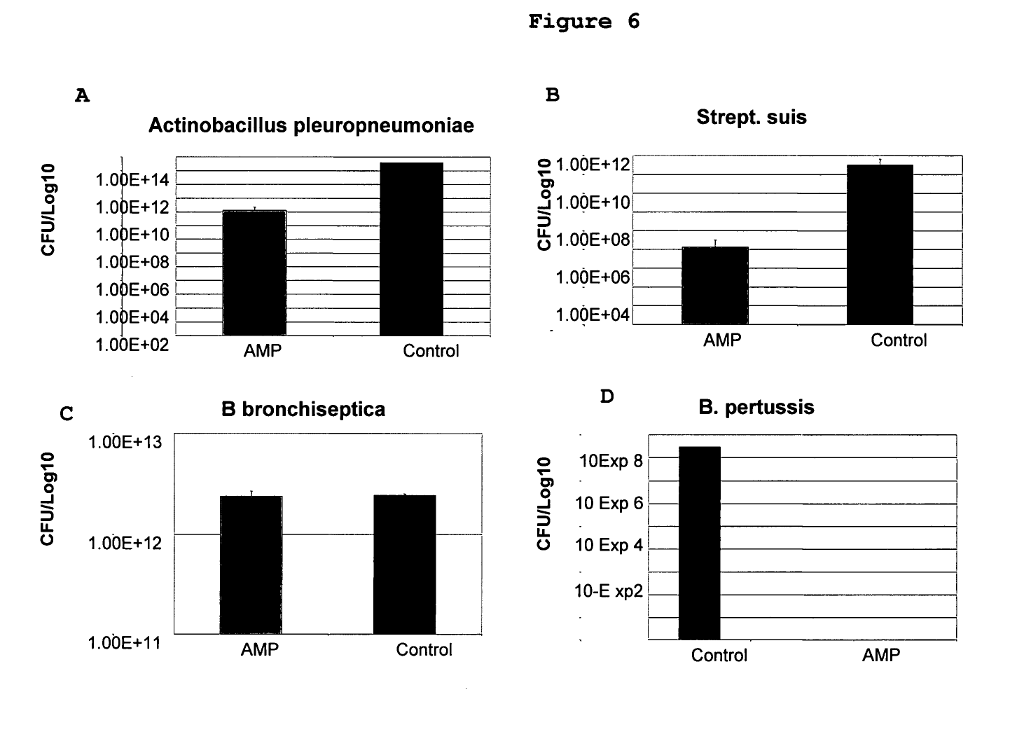

The inhibitory activity of pBD-1 on the Gram-negative bacteria Actinobacillus pleuropneumoniae, Bordetella bronchiseptica, Streptococcus suis and E. coli was also examined. FIGS. 6A-6B show the results on A. pleuropneumoniae, S. suis, B. bronchiseptica and B. pertussis. The results for E. coli are not shown. Inhibitory activity was observed against all of these organisms except B. bronchiseptica.

C. Effects of Varying Conditions on Bactericidal Activity

The effect of the SS medium on bactericidal activity of pBD-1 was tested by diluting the SS media in PBS. SS medium has comparable concentrations of Ca²⁺, Mg²⁺, and K⁺ to human lung secretions. It is possible that in vivo concentrations of ions such as Ca²⁺, Mg²⁺ or K⁺ as well as tissue proteins may interfere with antimicrobial activity of pBD-1 (Boman et al. (2003) J. Intern. Med. 254:197-215; Bowdish et al. (2005) Curr. Protein Pept. Sci. 6:35-51). In diluted medium, pBD-1 exhibited an even stronger bactericidal activity against B. pertussis in a shorter period of time (FIG. 5B). In addition, the effect of varying serum conditions in vitro was tested by adding 10-50% rabbit serum to the medium in order to simulate physiological conditions. Even very high serum concentrations (50%) did not inhibit the antimicrobial activity of pBD-1. Thus, these results provide additional support for the hypothesis that the expression of pBD-1 at the respiratory surfaces is crucial for providing protection against infection with B. pertussis.

### EXAMPLE 2

### Specificity of Antimicrobial Peptides

A. Bactericidal Activity of pBD-1 against B. pertussis and B. bronchiseptica

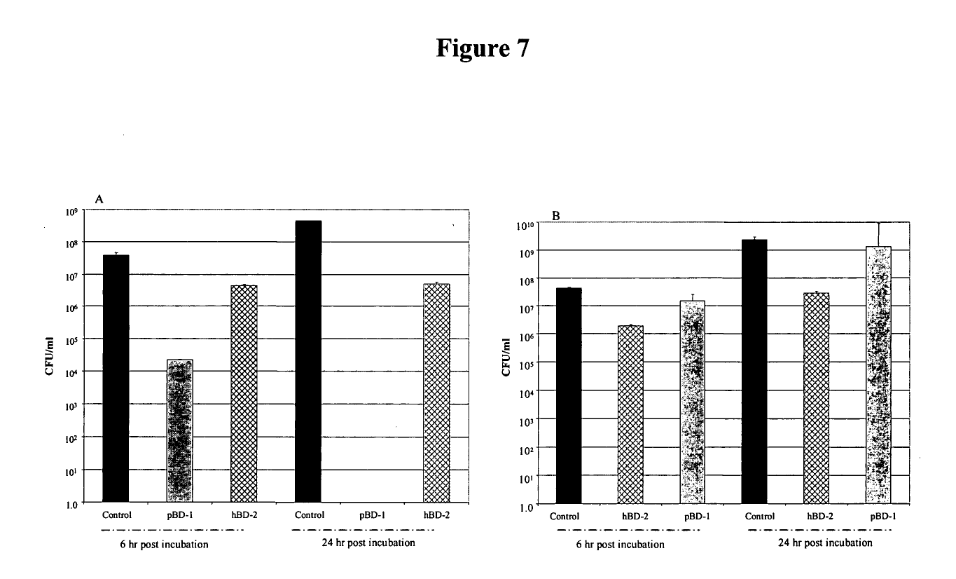

The bactericidal activity of pBD-1 against an experimentally introduced human pathogen, B. pertussis, and a natural pathogen of pigs, B. bronchiseptica, was compared in vitro using 40 μg/ml of pBD-1. The growth of B. pertussis was inhibited by 40% after 6 hours and by 100% after 24 hours (FIG. 7A). In contrast, the growth of B. bronchiseptica was unaffected at these time points (FIG. 7B). In addition, B. bronchiseptica was resistant to pBD-1 even at very high concentrations (160 μg/ml) and for up to 48 hours incubation. Thus, these results indicate that B. bronchiseptica, a natural pathogen of pigs, has evolved a means of evading the porcine innate immune system.

B. Bactericidal Activity of hBD-2

The bactericidal activity of hBD-2 against both B. pertussis and B. bronchiseptica was measured. Although, BD-2 is the human defensin most homologous to pBD-1, even at concentrations greater than 80 μg/ml, hBD-2 had no bactericidal activity and only a bacteriostatic effect against both B. pertussis and B. bronchiseptica in vitro (FIG. 7A). These results suggest that both B. pertussis and B. bronchiseptica have evolved in their respective host environments and that both have developed strategies to evade the innate immune defense.

### EXAMPLE 3

### Bactericidal Activity of BAL Fluid from Piglets

A. Bactericidal Activity of BALs against B. pertussis

Bactericidal activity of bronchoalveolar lavage (BAL) fluid obtained from non-infected piglets was analyzed against B. pertussis in vitro. Non-infected piglets, either 4-5 weeks old or newborn piglets (6 hours old, colostrum-deprived or colostrum-fed), were euthanized and BALs were collected in Stainer-Scholte (SS) medium. The BAL fluid was obtained by filling the lungs with 10 ml of SS medium and withdrawing as much fluid as possible (this procedure was performed once). Alveolar macrophages and other cells were removed by centrifugation at 500 g for 10 minutes. BALs (290 μl) were co-cultured in microtiter plates with 10 μl bacterial suspension containing 5-7×10⁶ CFU of B. pertussis at 37° C. Supernatants were plated onto BG agar plates at different time points to evaluate the number of viable bacteria. The sensitivity of B. pertussis to synthetically derived pBD-1 and human beta-defensin 2 (hBD-2) was assayed by co-culturing appropriate concentrations of pBD-1 or hBD-2 in SS-medium (280 μl) with 5-7×10⁶ CFU (10 μl) bacteria. Plates were incubated at 37° C. for 2, 6, 18 and 24 hours. The same procedures were followed to investigate the inhibitory effects of BAL, pBD-1 and hBD-2 against B. bronchiseptica in vitro.

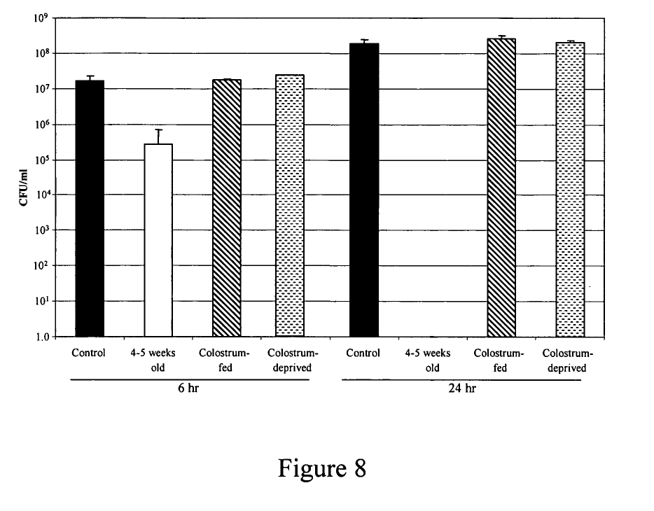

BALs from either newborn piglets (colostrum-fed/colostrum-deprived) or piglets 4-5 weeks old were co-cultured (total volume of 290 μl) with 5-7×10⁶ CFU B. pertussis in SS-medium (FIG. 8). Compared to the medium control, there was a significant time dependent reduction in the number of viable bacteria in BALs from piglets 4-5 weeks old. At 6 hours of incubation, bacterial numbers were reduced by 1.5 logs and after 24 hours, no viable bacteria were isolated from wells co-cultured with the BALs obtained from piglets 4-5 weeks old. In contrast, regardless of whether the BALs were obtained from colostrum-fed or colostrum-deprived newborn piglets, inhibitory effects against the growth of B. pertussis were not observed (FIG. 8). These results confirm that BALs obtained from older animals contain antimicrobial components that may be associated with in vivo protection against B. pertussis. Furthermore, these results confirm that the presence of colostrum derived S-IgA in BAL did not have any effect on bacterial growth or interfere with assays.

B. Salt-Dependence of BAL Antibacterial Activity against B. pertussis

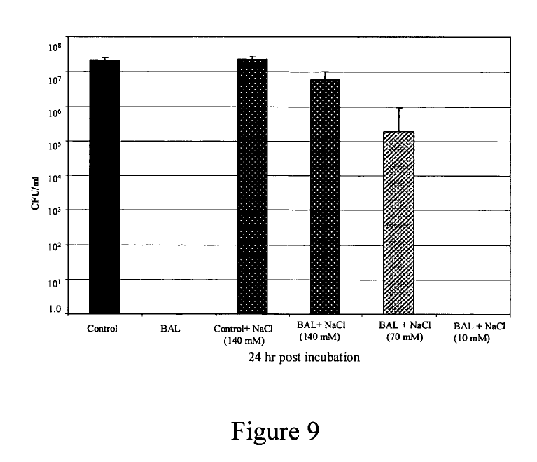

The antimicrobial activity of many cationic peptides including beta-defensins is greatly affected by higher salt concentrations (Bals, et al. (1998a) Infect. Immun. 66:1225-1232; Bals et al. (1998b) J. Clin. Invest. 102:874-880). For example, it has been reported that an elevated concentration of NaCl in the airway surface fluid of patients with cystic fibrosis inactivates the antimicrobial activity of defensins and predisposes the host to a wide range of infections (Bals et al. 1998b, supra). In order to determine whether the observed bactericidal activity of BAL was due to the presence of cationic peptides, 10-140 mM NaCl was added to the BALs. As shown in FIG. 9, 140 mM salt completely and 70 mM salt partially blocked the growth inhibitory effect of BAL against B. pertussis. However, 10 mM NaCl did not inhibit the antibacterial activity of BALs (FIG. 9). Thus, these results demonstrate that the observed antimicrobial effect was reversed by addition of high salt concentration to the culture and therefore the antimicrobial activity of BALs obtained from piglets 4-5 weeks old could be due to the presence of cationic peptides, such as pBD-1, in lung secretions.

C. Conclusion

Defensins exhibit broad spectrum antimicrobial activity in vitro against bacterial, fungal and viral pathogens. BALs from older animals displayed significant bactericidal activity against B. pertussis in vitro, whereas BALs from either colostrum-fed or colostrum-deprived newborn piglets did not demonstrate any anti-B. pertussis activity. Substantial reduction of the observed activity of BALs by high NaCl concentrations confirmed the involvement of cationic peptides in antimicrobial action as described by others (Bals et al. 1998a, supra; Miyasaki et al. (1990) Infect. Immun. 58:3934-3940). This was further confirmed by the observation that the antimicrobial activity of the synthetically derived pBD-1 against B. pertussis was significantly reduced by the addition of salt.

### EXAMPLE 4

### Antimicrobial Activity of pBD-1 In Vivo

The antimicrobial activity of pBD-1 was tested in vivo by delivery of pBD-1 into the lungs of newborn piglets prior to challenge with B. pertussis. Pregnant sows, Landrace, were purchased from Saskatoon Prairie Swine Centre, University of Saskatchewan. Sows were farrowed out by i.m. injection of prostaglandin (Planate). Three to four day old colostrum-fed piglets were anaesthetized with isoflurane. 500 μg of pBD-1 dissolved in 1.5 ml PBS was delivered to the lung using a tube placed through the trachea in front of the bifurcation which marks the beginning of the lung (craniodorsal of bifurcation). A volume of 1.5 ml was delivered. Following the delivery of the pBD-1, all piglets were challenged with 5×10⁹ cfu bacteria (n=10) by the same procedure. Control animals received saline instead of pBD-1 and were subsequently challenged. Piglets were kept with their sows within the same room in separated pens. Piglets were monitored twice a day.

Co-delivery of pBD-1 into the lungs of newborn piglets resulted in protection against infection with 5×10⁹ c.f.u. B. pertussis. Whereas all control animals displayed fever and respiratory symptoms, all pBD-1 treated animals displayed no clinical symptoms. Indeed, treatment with only 500 μg of pBD-1 at the time of challenge resulted in complete protection of infected piglets as demonstrated by the total absence of clinical symptoms and pathological alterations at 2, 4, 7 and 10 days post infection. Those piglets that were treated with pBD-1 indicated early recovery by showing either minor or no lesions in the histopathology of lung tissues compared with challenged control piglets which did not receive defensin.

In contrast, severe subacute hemorrhagic and necrotizing pneumonia was found in control (PBSA-treated) piglets. Lesions were characterized by severe cellular infiltrations (neutrophils and macrophages) into the alveolar spaces, around the bronchioles and in the walls of blood vessels with severe alveolar hemorrhagic, congestion, edema and focal bronchiolar necrosis causing suppurative and histiocytic pleuropneumonia and bronchointerstitial pneumonia.

In addition, the number of bacteria in BAL fluid and lung lesions was examined over a 10 day period. The BAL fluid was obtained by filling the lungs with 15 ml of SS-medium and withdrawing as much fluid as possible. To quantify the presence of B. pertussis in the BAL, fluid samples were centrifuged to remove debris and host cells; supernatant and dilutions thereof were plated onto BG plates and incubated at 37° C. for up to a week. To determine the number of bacteria within tissues, lesions were excised, weighed, homogenized and plated onto BG agar plates.

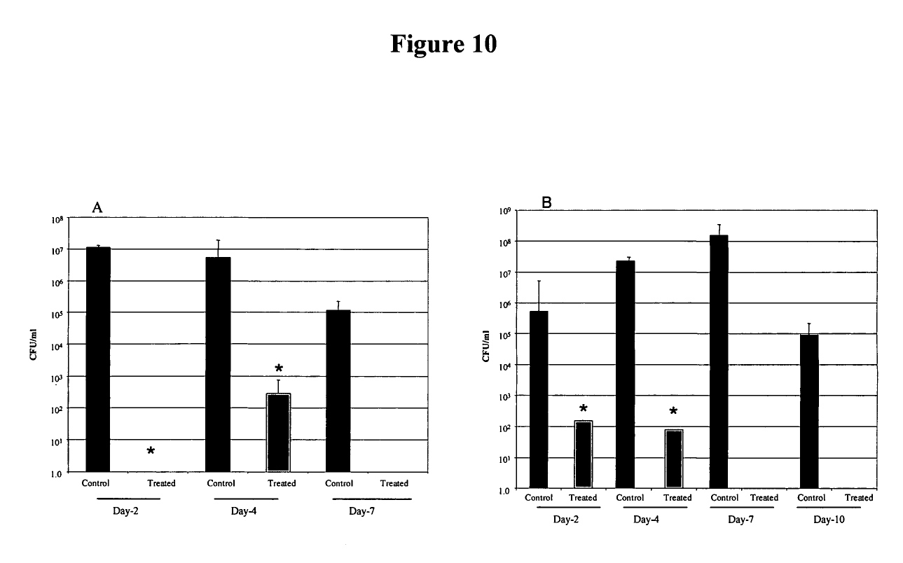

The numbers of isolated bacteria in BALs collected from pBD-1-treated animals were significantly reduced (p<0.0001) at days 2, 4 and 7 days post infection. (FIG. 10A). In addition, significantly lower numbers of bacteria (p<0.005) were found in homogenized lung tissues of treated animals at days 2, 4, 7, and 10 days post infection (FIG. 10B). In contrast, BALs and lung tissues collected from control animals contained between 10⁵ and 10⁸ CFU/g tissue at these days respectively. Thus, the results demonstrate that defensins play an essential role in mediating protection against pertussis. Furthermore, the results clearly demonstrate that treatment with chemically synthesized AMPs may represent a possible means of treating this important disease in young children and infants.

### EXAMPLE 5

### Analysis of pBD-1 Expression in Piglets of Different Ages

While older piglets are fully protected against infection with B. pertussis, newborn piglets are susceptible to the disease. To determine if the level of defensin increases with age, the expression of pBD-1 in tongue, respiratory epithelium (nasal mucosa, trachea, and lung), and the small intestine obtained from newborn piglets and piglets 4-5 weeks old was analyzed.

Transcripts were detected by reverse transcriptase-polymerase chain reaction (RT-PCR). Total RNA was extracted from porcine tissues by the acid guanidium thiocyanate/phenolchloroform method using a total RNA isolation reagent (Trizol Reagent, GIBCO BRL). RNA was reverse transcribed to cDNA, using Oligo (dT) primer (Sigma-Aldrich), according to the manufacturer's instructions. RT products were amplified using a PCR kit (RED Taq, Ready Mix PCR Reaction Mix, Sigma-Aldrich) and a thermal cycler (Gene Amp PCR System 9700, Applied Biosystems, Singapore). The primers used to detect a 287 bp cDNA sequence of pBD-1 in porcine tissues were a sense primer 5′-TCCCATGAGACTCCACCGCCTCCT-3′ (SEQ ID NO:3) and an antisense primer 5′-TTCGAGCAGCTTCTGAGCCATATCTGT (SEQ ID NO:4). PCR was performed as follows: denaturation at 95° C. for 1 min, followed by 30 cycles of denaturation at 94° C. for 15 seconds, annealing at 60° C. for 30 seconds, and extension at 72° C. for 30 seconds, followed by a final extension at 72° C. for 7 minutes. The PCR products were visualized by electrophoresis on 1.5% agarose gel containing 0.5 μg/ml ethidium bromide.

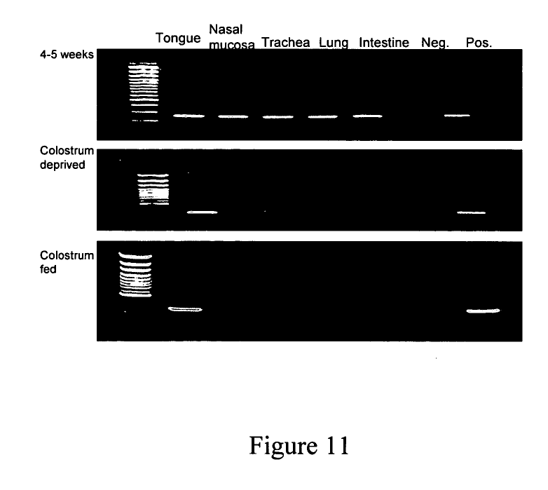

Expression of pBD-1 was detected in all analyzed tissues in piglets 4-5 weeks old, including tongue, intestine, nasal mucosa, trachea and lung tissues (FIG. 11). In contrast, expression of pBD-1 in tissues obtained from newborn piglets was detected only in the tongue epithelium. Without being bound by a particular theory, the differences observed in susceptibility to infections between newborn piglets and older pigs may be due in part to the differing levels of secretion of defensin in the lung. The expression of pBD-1 in older piglets contributed to the resistance against infection with B. pertussis. In addition, HPLC analysis of BAL fluid revealed the presence of pBD-1 in BALs collected from older piglets but not from newborn animals. Thus, protection against infection is associated with the presence of pBD-1 at the upper respiratory surfaces.

### EXAMPLE 6

### Transmission Electron Microscopy

To determine the potential mechanism of action of pBD-1, transmission electron microscopy (TEM) was employed to evaluate the effect of pBD-1 on the bacterial membrane of B. pertussis and B. bronchiseptica following treatment with pBD-1. pBD-1 was added at a final concentration of 20 μg/ml or 40 μg/ml to 5-9×10⁹ CFU bacteria and incubated for 60 minutes at 37° C. Bacteria were washed with PBS, fixed with an equal volume of 5% glutaraldehyde in 0.1 M sodium cacodylate buffer (pH 7.4), and centrifuged at 5000 g for 5 minutes. Cells were resuspended in 1% agarose and pelleted. The bacterial pellets were stored in the fixative (2.5% glutaraldehyde) at 4° C. for 2 hours. The samples were dehydrated and en bloc stained with uranyl acetate in a series of ethanol concentrations and then embedded in Epon/Araldite. Thin sections were cut using an ultramicrotome with a diamond knife, mounted on specimens grids and stained with uranyl acetate and lead citrate before being examined on a transmission electron microscope (Philips 410LS) operating at 80 kV and at magnification ranging from 24000-55000×.

The exposure of B. pertussis to two different concentrations (20 and 40 μg/ml) of pBD-1 resulted in a substantial morphological damage to the cell surface of bacteria as shown by SEM. Untreated bacteria displayed a rough brighter surface with no apparent perforation and cellular debris. In contrast, B. pertussis exposed to pBD-1 exhibited a wide range of morphological abnormalities. These included disruption of the cell surface, disappearance of the cell membrane, perforation and breakage in the cell membrane and lysis of bacteria. The effects of pBD-1 on B. bronchiseptica at significant greater concentrations (80 μg/ml) did not result in major ultrastructural abnormalities or discrete holes compared with non-treated B. bronchiseptica bacteria.

In conclusion, the above results, taken together, demonstrate a protective role for pBD-1 against B. pertussis both in vitro and in vivo and provide a new approach to therapy of this disease.

Thus, novel methods for treating and preventing infectious diseases are disclosed. Although preferred embodiments of the subject invention have been described in some detail, it is understood that obvious variations can be made without departing from the spirit and the scope of the invention as defined by the claims.

What is claimed is:

1. A method of treating or preventing a microbial infection, said method comprising administering to a vertebrate subject a pharmaceutically effective amount of a composition comprising a porcine β-defensin.

2. The method of claim 1, wherein the microbial infection is caused by a microbe selected from the group consisting of a bacteria, a virus, a fungus and a parasite.

3. The method of claim 2, wherein the microbial infection is caused by a Gram-negative bacterium.

4. The method of claim 1, wherein the porcine β-defensin is a porcine β-defensin-1 (pBD-1).

5. The method of claim 4, wherein the pBD-1 comprises contiguous amino acid residues 23-64 of the sequence of SEQ ID NO:2, or an amino acid sequence with at least 75% sequence identity thereto.

6. The method of claim 5, wherein the pBD-1 comprises an amino acid sequence with at least 85% identity to the contiguous amino acid sequence of residues 23-64 of SEQ ID NO:2.

7. The method of claim 5, wherein the pBD-1 comprises an amino acid sequence with at least 95% identity to the contiguous amino acid sequence of residues 23-64 of SEQ ID NO:2.

8. The method of claim 5, wherein the pBD-1 comprises the contiguous amino acid sequence of residues 23-64 of SEQ ID NO:2.

9. The method of claim 8, wherein the pBD-1 comprises the contiguous amino acid sequence of residues 1-64 of SEQ ID NO:2, or an amino acid sequence with at least 75% sequence identity thereto.

10. The method of claim 9, wherein the pBD-1 comprises an amino acid sequence with at least 85% identity to the contiguous amino acid sequence of residues 1-64 of SEQ ID NO:2.

11. The method of claim 9, wherein the pBD-1 comprises an amino acid sequence with at least 95% identity to the contiguous amino acid sequence of residues 1-64 of SEQ ID NO:2.

12. The method of claim 9, wherein the pBD-1 comprises the contiguous amino acid sequence of residues 1-64 of SEQ ID NO:2.

13. The method of claim 3, wherein the Gram-negative bacterium is selected from the group consisting of Actinobacillus pleuropneumoniae, Bordetella pertussis Bordetella parapertussis, Streptococcus suis and Escherichia coli.

14. The method of claim 13, wherein the Gram-negative bacterium is Bordetella pertussis.

15. A method of treating or preventing Bordetella pertussis infection, said method comprising administering to a mammalian subject a pharmaceutically effective amount of a composition comprising a porcine β-defensin-1 (pBD-1), wherein the pBD-1 comprises the contiguous amino acid sequence of residues 1-64 of SEQ ID NO:2, or an amino acid sequence with at least 75% sequence identity thereto.

16. The method of claim 15, wherein the pBD-1 comprises an amino acid sequence with at least 85% identity to the contiguous amino acid sequence of residues 1-64 of SEQ ID NO:2.

17. The method of claim 15, wherein the pBD-1 comprises an amino acid sequence with at least 95% identity to the contiguous amino acid sequence of residues 1-64 of SEQ ID NO:2.

18. The method of claim 15, wherein the pBD-1 comprises the contiguous amino acid sequence of residues 1-64 of SEQ ID NO:2.

19. A method of enhancing an immunological response to an antigen present in a composition for treating or preventing a microbial infection, said method comprising administering to a vertebrate subject said composition and a pharmaceutically effective amount of a porcine β-defensin.

20. The method of claim 19, wherein the microbial infection is caused by a microbe selected from the group consisting of a bacteria, a virus, a fungus and a parasite.

21. The method of claim 20, wherein the microbial infection is caused by a Gram-negative bacterium.

22. The method of claim 19, wherein the porcine β-defensin is a porcine β-defensin-1 (pBD-1).

23. The method of claim 22, wherein the pBD-1 comprises the contiguous amino acid sequence of residues 23-64 of SEQ ID NO:2, or an amino acid sequence with at least 75% sequence identity thereto.

24. The method of claim 23, wherein the pBD-1 comprises an amino acid sequence with at least 85% identity to the contiguous amino acid sequence of residues 23-64 of SEQ ID NO:2.

25. The method of claim 23, wherein the pBD-1 comprises an amino acid sequence with at least 95% identity to the contiguous amino acid sequence of residues 23-64 of SEQ ID NO:2.

26. The method of claim 23, wherein the pBD-1 comprises the contiguous amino acid sequence of residues 23-64 of SEQ ID NO:2.

27. The method of claim 26, wherein the pBD-1 comprises the contiguous amino acid sequence of residues 1-64 of SEQ ID NO:2, or an amino acid sequence with at least 75% sequence identity thereto.

28. The method of claim 27, wherein the pBD-1 comprises an amino acid sequence with at least 85% identity to the contiguous amino acid sequence of residues 1-64 of SEQ ID NO:2.

29. The method of claim 27, wherein the pBD-1 comprises an amino acid sequence with at least 95% identity to the contiguous amino acid sequence of residues 1-64 of SEQ ID NO:2.

30. The method of claim 27, wherein the pBD-1 comprises the contiguous amino acid sequence of residues 1-64 of SEQ ID NO:2.

31. The method of claim 21, wherein said antigen is a Bordetella pertussis antigen.

32. A method of enhancing an immunological response to a Bordetella pertussis antigen, said method comprising administering to a vertebrate subject a vaccine composition comprising said Bordetella pertussis antigen, and administering a pharmaceutically effective amount of a porcine β-defensin-1 (pBD-1), wherein the pBD-1 comprises the contiguous amino acid sequence of residues 1-64 of SEQ ID NO:2, or an amino acid sequence with at least 75% sequence identity thereto.

33. The method of claim 32, wherein the pBD-1 comprises an amino acid sequence with at least 85% identity to the contiguous amino acid sequence of residues 1-64 of SEQ ID NO:2.

34. The method of claim 32, wherein the pBD-1 comprises an amino acid sequence with at least 95% identity to the contiguous amino acid sequence of residues 1-64 of SEQ ID NO:2.

35. The method of claim 32, wherein the pBD-1 comprises the contiguous amino acid sequence of residues 1-64 of SEQ ID NO:2.

36. A method of modulating an immunological response to a Gram-negative bacterium, said method comprising administering to a vertebrate subject a pharmaceutically effective amount of a porcine β-defensin.

37. The method of claim 36, wherein the porcine β-defensin is a porcine β-defensin-1 (pBD-1).

38. The method of claim 37, wherein the pBD-1 comprises the contiguous amino acid sequence of residues 23-64 of SEQ ID NO:2, or an amino acid sequence with at least 75% sequence identity thereto.

39. The method of claim 37, wherein the pBD-1 comprises an amino acid sequence with at least 85% identity to the contiguous amino acid sequence of residues 23-64 of SEQ ID NO:2.

40. The method of claim 37, wherein the pBD-1 comprises an amino acid sequence with at least 95% identity to the contiguous amino acid sequence of residues 23-64 of SEQ ID NO:2.

41. The method of claim 37, wherein the pBD-1 comprises the contiguous amino acid sequence of residues 23-64 of SEQ ID NO:2.

42. The method of claim 41, wherein the pBD-1 comprises the contiguous amino acid sequence of residues 1-64 of SEQ ID NO:2, or an amino acid sequence with at least 75% sequence identity thereto.

43. The method of claim 42, wherein the pBD-1 comprises an amino acid sequence with at least 85% identity to the contiguous amino acid sequence of residues 1-64 of SEQ ID NO:2.

44. The method of claim 42, wherein the pBD-1 comprises an amino acid sequence with at least 95% identity to the contiguous amino acid sequence of residues 1-64 of SEQ ID NO:2.

45. The method of claim 42, wherein the pBD-1 comprises the contiguous amino acid sequence of residues 1-64 of SEQ ID NO:2.

46. The method of claim 36, wherein said antigen is a Bordetella pertussis antigen.
